(19) United States
(12) Patent Application Publication
(10) Pub. No.: US 2024/0358497 A1
(43) Pub. Date: Oct. 31, 2024

(54) CAPSULAR STABILIZERS AND METHODS FOR PREVENTING ROTATION OF INTRACULAR LENS AND FIXATING THE SAME TO THE ANTERIOR CAPSULE

(71) Applicant: ELIACHAR TECHNOLOGIES DEVELOPMENTS LTD., Haifa (IL)

(72) Inventors: Ehud ASSIA, Tel Aviv (IL); Eliahu ELIACHAR, Haifa (IL); Nir LILACH, Kfar Yehoshua (IL); Ram GROSSFELD, Ramat Yishay (IL); Yaniv SIBIRSKI, Yokneam Illit (IL)

(21) Appl. No.: 18/291,299

(22) PCT Filed: Jun. 28, 2022
(86) PCT No.: PCT/IL2022/050693

§ 371 Date: Jan. 23, 2024

Related U.S. Application Data
(60) Provisional application No. 63/225,063, filed on Jul. 23, 2021.

(51) Int. Cl. A61F 2/16

(52) U.S. Cl. CPC A61F 2/1613; A61F 2002/1689; A61F 2002/169; A61F 2220/0016

(57) ABSTRACT

The present invention discloses capsular stabilizers, added to or contained in an intraocular lens (IOL). The present invention also discloses IOLs configured to be affixed to capsular stabilizers, kits thereof, and methods for (i) increasing postoperative rotation stability of an IOL and preferably a toric IOL; and (ii) stabilizing an IOL within the anterior capsule for any indication, including in cases at which the posterior capsule is absent or ruptured.

### FIELD OF THE INVENTION

The present invention generally relates to capsular stabilizers, added to or contained in an intraocular lens (IOL), a kit for adding capsular stabilizers to an IOL and preferably a toric IOL, and methods for (i) increasing postoperative rotational stability of an IOL; and (ii) stabilizing an IOL to the anterior capsule of the eye for any indication, including in cases at which the posterior capsule is absent or ruptured.

### BACKGROUND OF THE INVENTION

Cataract surgery is one of the most successful and most frequently performed surgical procedures in the world. Each year, millions of people achieve a dramatic improvement in their visual function thanks to this procedure. With the increasing proportion of the US population reaching their retirement years, there is expected to be a doubling of the demand for cataract surgery over the next twenty years from 3.3 million to over 6 million annually. Worldwide it is estimated that about 30 million people undergo cataract surgery. This increased demand will require more ophthalmologists to be trained to perform cataract surgery as well as each trained ophthalmologist performing an increased number of cataract surgeries each year.

It has been estimated that 15% to 29% of patients with cataract have more than 1.50 diopters (D) of preexisting astigmatism and, since the rate of astigmatism increases with age, a clinically significant astigmatism is present in approximately one half of the patients undergoing cataract surgery. Reducing this preexisting astigmatism may further improve visual outcomes after cataract surgery. Astigmatism can be reduced or eliminated with several techniques, including selective positioning of the phacoemulsification incision, corneal relaxing incisions, limbal relaxing incisions, or excimer laser keratectomy. All these methods have limitations including accuracy and predictability, the amount of astigmatism that can be treated and long-term mechanical instability, and postoperative outcomes are subject to many variables such as age; magnitude of the astigmatism; and incision number, depth, and length.

The current optimal means of treating astigmatism is by using toric intraocular lenses (IOLs) that are positioned at a specific, predetermined axis. The most common complication of a toric lenses is rotational instability and lens rotation that can reduce the cylindric effect of the IOL. The maximum incidence of IOL rotation occurs in the first hour to 10 days after implantation of the IOL. Even a small degree of rotation of a toric IOL from its intended axis can result in large reduction of astigmatic correction and patient dissatisfaction. For example, a deviation of 10 degrees minimizes the potential correction by approximately 35%, in other words every degree of rotation reduces the astigmatic correction by 3.3-3.5%. When a toric IOL rotates 30 degrees, cylinder power is completely lost.

Hence, inaccuracies during measurement of the various ocular geometries, inaccuracies during surgery and postsurgical effects (such as surgical trauma and wound healing processes) cause positioning errors of the implanted IOL that limit the achievable visual acuity. Rotation of the toric lens is not uncommon and most usually occurs spontaneously, most often during the first day or even hours post-surgery, before capsule contraction and fibrosis maintain rotational stability. Positioning errors with respect to the optical axis mainly cause defocusing while tilt and decentration of the IOL will result in induced astigmatism and coma errors.

Proper intraocular lens (IOL) position and centration affects the visual outcomes of patients undergoing cataract surgery, and is crucial in toric IOL implantation. IOL malposition can lead to patient dissatisfaction, the dissatisfaction resulting from a decrease in the quality of vision due to the refractive errors and higher-order aberrations; see Assia, Ehud I et al. “The Effect on Post-Operative IOL Centration by Manual Intraoperative Centration versus Auto-Centration.” Clinical ophthalmology (Auckland, N.Z.) vol. 14 3475-3480. 23 Oct. 2020.

It is hence a long felt need for (i) increasing postoperative rotation stability of an IOL's rotational angle in order to maintain correction of the unique optical distortion to the patient's eye; and (ii) stabilizing the IOL to the anterior capsule.

### SUMMARY OF THE INVENTION

It is thus an object of the invention to disclose an IOL fixating means to enable fixating the IOL and preferably a toric IOL to the anterior capsule to (i) increasing postoperative rotation and increase the stability; and (ii) enabling IOL attachment/fixation to the anterior capsule in order to prevent the lens from falling into the eye for any indication, including in cases at which the posterior capsule is absent or ruptured, the IOL having a main longitudinal axis B:B, an anterior surface of a main plane X:Y and a posterior surface, the IOL comprising a lens interconnected to at least one haptic, characterized by a plurality of fasteners affixable onto said anterior surface along said main longitudinal axis B:B; when affixed, heads of said plurality of fasteners protrude from said anterior surface X:Y so that a gap of a predefined size and shape is provided.

It is another object of the invention to disclose the IOL as defined in any of the above, wherein the lens is selected from a group consisting of a toric lens and a non-toric lens.

It is another object of the invention to disclose the IOL as defined in any of the above, wherein said at least one haptic is selected from a group consisting of a plate-type haptic and a loop-type haptic.

It is another object of the invention to disclose the IOL as defined in any of the above, wherein said plurality of fasteners comprises n fasteners, n being equal to or greater than 2.

It is another object of the invention to disclose the IOL as defined in any of the above, wherein n is in a range selected from a group consisting of 2 to 4, 6 to 8, and more than 10.

It is another object of the invention to disclose the IOL as defined in any of the above, wherein said IOL further comprises a member of a group consisting of (i) at least one vertical hole, perpendicular to surface X:Y; (ii) at least one horizontal hole parallel to surface X:Y; (iii) at least one vertically protruding structure, positioned perpendicular to surface X:Y; (iv) at least one horizontally protruding structure, positioned perpendicular to surface X:Y, and any combination thereof.

It is another object of the invention to disclose the IOL as defined in any of the above, wherein at least one of said plurality of fasteners is selected from a group consisting of a symmetric snap, an asymmetric snap, a jeans-button anchor, a hook-containing member, a loop-containing member, a hot-melt snap, a toothed clasp, a hook, a peg, a catch, a spring fastener and any combination thereof.

It is another object of the invention to disclose the IOL and preferably a toric IOL for (i) increasing postoperative rotation stability; and (ii) enabling stabilization to the anterior capsule for any indication, including in cases at which the posterior capsule is absent or ruptured, having a main longitudinal axis B:B, an anterior surface of a main plane X:Y and a posterior surface, comprising a lens interconnected to at least one haptic, characterized by a plurality of clasp-like snaps affixable onto said anterior surface along said main longitudinal axis B:B; when each of said a plurality of clasp-like snaps is affixed, a neck of each of said plurality of clasp-like snaps either forms a groove or comprises a groove of a predefined size and shape.

It is another object of the invention to disclose the IOL as defined in any of the above, wherein said lens is either a toric lens or a non-toric lens; each of said at least one haptic is selected from a group consisting of a plate-type haptic, a loop-type haptic and any combination thereof.

It is another object of the invention to disclose the IOL as defined in any of the above, wherein said IOL comprises n of said plurality of clasp-like snaps, n being equal to or greater than 2.

It is another object of the invention to disclose the IOL as defined in any of the above, wherein n is in a range selected from a group consisting of 2 to 4, 6 to 8, and more than 10.

It is another object of the invention to disclose the IOL as defined in any of the above, wherein said IOL further comprises a member of a group consisting of (i) at least one vertical hole; (ii) at least one horizontal hole; (iii) at least one vertically protruding structure; (iv) at least one horizontally protruding structure, and any combination thereof.

It is another object of the invention to disclose the IOL as defined in any of the above, additionally comprising a plurality of clasp-like snaps affixable onto said anterior surface along said main longitudinal axis B:B.

It is another object of the invention to disclose the IOL as defined in any of the above, wherein at least one of said plurality of clasp-like snaps is selected from a group consisting of a toothed clasp, a clip-like fastener, a clamp-like fastener, a spring fastener, a pliers-like clasp and any combination thereof.

It is another object of the invention to disclose a vertical capsular stabilizer (VCS) useful for (i) increasing postoperative rotation stability of IOL and preferably a toric IOL; and (ii) enabling stabilization of an IOL to the anterior capsule for any indication, including in cases at which the posterior capsule is absent or ruptured, having a main longitudinal axis B:B, an anterior surface of a main plane X:Y and a posterior surface, comprising a lens interconnected to at least one haptic; said VCS is affixable perpendicular to said anterior surface along said main longitudinal axis B:B; when affixed, a head of said VCS protrudes from said anterior surface X:Y so that a gap of a predefined size and shape is provided.

It is another object of the invention to disclose the VCS as defined in any of the above, wherein said lens is either a toric lens or a non-toric lens; said at least one haptic is selected from a group consisting of a plate-type haptic and a loop-type haptic

It is another object of the invention to disclose the VCS as defined in any of the above, wherein said IOL comprises n of said at least one VCS, n being equal to or greater than 2.

It is another object of the invention to disclose the VCS as defined in any of the above, wherein n is in a range selected from a group consisting of 2 to 4, 6 to 8, and more than 10.

It is another object of the invention to disclose the VCS as defined in any of the above, wherein the VCS is selected from a group consisting of a snap, a jeans-button anchor, a hook-containing member, a loop-containing member, a hot-melt snap, a toothed clasp, a spring fastener and any combination thereof.

It is another object of the invention to disclose a horizontal capsular stabilizer (HCS) useful for at least one of (i) increasing postoperative rotation stability of an IOL and preferably a toric IOL; and (ii) enabling stabilization of an IOL to the anterior capsule for any indication, including in cases at which the posterior capsule is absent or ruptured, having a main longitudinal axis B:B, an anterior surface of a main plane X:Y and a posterior surface, comprising a lens interconnected to at least one haptic; said HCS is affixable parallel to said anterior surface along said main longitudinal axis B:B; when affixed, a head of said HCS either forms a groove or comprises a groove positioned perpendicular to said anterior surface X:Y so that a gap of a predefined size and shape is provided.

It is another object of the invention to disclose the HCS as defined in any of the above, wherein said lens is either a toric lens or a non-toric lens; said at least one haptic being selected from a group consisting of a plate-type haptic, a loop-type haptic and any combination thereof;

It is another object of the invention to disclose the HCS as defined in any of the above, wherein said IOL comprises n of said at least one HCS, n being equal to or greater than 2.

It is another object of the invention to disclose the HCS as defined in any of the above, wherein n is in a range selected from a group consisting of 2 to 4, 6 to 8, and more than 10.

It is another object of the invention to disclose the HCS as defined in any of the above, wherein the HCS is selected from a group consisting of a snap, a jeans-button anchor, a hook-containing member, a loop-containing member, a hot-melt snap, a toothed clasp, a spring fastener and any combination thereof.

It is another object of the invention to disclose, an IOL preferably a toric IOL, characterized by at least one of (i) an increased postoperative rotation stability; and (ii) an increased stabilization to the anterior capsule for any indication, including in cases at which the posterior capsule is absent or ruptured, having a main longitudinal axis B:B, an anterior surface of a main plane X:Y and a posterior surface, comprising a lens interconnected to at least one haptic, a vertical capsular stabilizer (VCS) reversibly affixable perpendicular to said anterior surface along said main longitudinal axis B:B; when affixed, a fastener head of said VCS protrudes from said anterior surface X:Y so that a gap of a predefined size and shape is provided, facing an opposite side of a center of said lens.

It is another object of the invention to disclose an IOL preferably a toric IOL, characterized by at least one of (i) an increased postoperative rotation stability; and (ii) an increased stabilization to the anterior capsule for any indication, including in cases at which the posterior capsule is absent or ruptured, having a main longitudinal axis B:B, an anterior surface of a main plane X:Y and a posterior surface, comprising a lens interconnected to at least one haptic, a horizontal capsular stabilizer (HCS) reversibly affixable on said anterior surface along said main longitudinal axis B:B; when affixed, a head of said stabilizer forms a groove positioned perpendicular to said anterior surface X:Y so that a gap of a predefined size and shape is provided, facing an opposite side of a center of said lens.

It is another object of the invention to disclose a kit, that adds Capsular Fasteners to an IOL that is then characterized by at least one of (i) an increased postoperative rotation stability; and (ii) an increased stabilization to the anterior capsule for any indication, including in cases at which the posterior capsule is absent or ruptured, said kit comprising an IOL having a main longitudinal axis B:B, an anterior surface of a main plane X:Y and a posterior surface, comprising a lens interconnected to at least one haptic; and a vertical capsular stabilizer (VCS) reversibly affixable perpendicular to said anterior surface along said main longitudinal axis B:B; when affixed, a fastener head of said VCS protrudes from said anterior surface X:Y so that a gap of a predefined size and shape is provided, facing an opposite side of a center of said lens.

It is another object of the invention to disclose a kit, that adds Capsular Fasteners to an IOL that is then characterized by at least one of (i) an increased postoperative rotation stability; and (ii) an increased stabilization to the anterior capsule for any indication, including in cases at which the posterior capsule is absent or ruptured, said kit comprising an IOL having a main longitudinal axis B:B, an anterior surface of a main plane X:Y and a posterior surface, comprising a lens interconnected to at least one haptic; and at least one horizontal capsular stabilizer (HCS) reversibly affixable on said anterior surface along said main longitudinal axis B:B; when affixed, a head of said stabilizer forms a groove positioned perpendicular to said anterior surface X:Y so that a gap of a predefined size and shape is provided, facing an opposite side of a center of said lens.

It is another object of the invention to disclose a method for at least one of (i) increasing postoperative rotation stability of an IOL and preferably a toric IOL; and (ii) stabilization of an IOL to the anterior capsule for any indication, including in cases at which the posterior capsule is absent or ruptured; said method comprising steps of: providing an IOL having a main longitudinal axis B:B, an anterior surface of a main plane X:Y and a posterior surface, comprising a lens interconnected to at least one haptic. Affixing at least one vertical capsular stabilizer (VCS) perpendicular to said anterior surface along said main longitudinal axis B:B; when affixed, a fastener head of said at least one VCS protrudes from said anterior surface X:Y so that a gap of a predefined size and shape is provided, facing an opposite side of a center of said lens; if astigmatism correction is required along radian A:A, ex situ affixing a plurality of said at least one VCS on said anterior surface along said main longitudinal axis B:B; so that heads of said plurality of said at least one VCS protrude from said anterior surface X:Y so that a gap of a predefined size and shape is provided, facing an opposite side of a center of said lens; implanting said IOL, said main longitudinal axis B:B positioned on an arbitrary radian; rotating in situ said IOL comprising said plurality of said at least one VCS so that said main longitudinal axis B:B is superimposed on said astigmatism radian A:A; and in situ positioning of the capsule peripheral circumferential edge over the gap to fixate the lens at the required angle, prevent rotation of the lens and enable fixation to the anterior capsule.

It is another object of the invention to disclose the method as defined in any of the above, wherein said lens is either a toric lens or a non-toric lens; said at least one haptic is selected from a group consisting of a plate-type haptic, a loop-type haptic and any combination thereof.

It is another object of the invention to disclose the method as defined in any of the above, wherein said IOL comprises n of said at least one VCS, n being equal to or greater than 2.

It is another object of the invention to disclose the method as defined in any of the above, wherein n is in a range selected from a group consisting of 2 to 4, 6 to 8, and more than 10.

It is another object of the invention to disclose the method as defined in any of the above, wherein said IOL further comprises a member of a group consisting of (i) at least one vertical hole, perpendicular to surface X:Y; (ii) at least one horizontal hole parallel to surface X:Y; (iii) at least one vertically protruding structure, positioned perpendicular to surface X:Y; (iv) at least one horizontally protruding structure, positioned perpendicular to surface X:Y, and any combination thereof.

It is another object of the invention to disclose the method as defined in any of the above, wherein said at least one VCS is selected from a group consisting of a symmetric snap, an asymmetric snap, a jeans-button anchor, a hook-containing member, a loop-containing member, a hot-melt snap, a toothed clasp, a spring fastener and any combination thereof.

It is still another object of the invention to disclose a method of preventing postoperative rotation of an IOL and preferably a toric IOL, comprising steps of providing an IOL having a main longitudinal axis B:B, an anterior surface of a main plane X:Y and a posterior surface, said IOL comprising a lens interconnected to at least one haptic; affixing at least one horizontal capsular stabilizer (HCS) on said anterior surface along said main longitudinal axis B:B; when affixed, a head of said at least one HCS either comprises a groove or forms a groove positioned perpendicular to said anterior surface X:Y so that a gap of a predefined size and shape is provided, facing an opposite side of a center of said lens; if astigmatism correction is required along radian A:A, ex situ affixing a plurality of said at least one VCS on said anterior surface along said main longitudinal axis B:B; so that heads of said plurality of said at least one HCS protrude from said anterior surface X:Y so that a gap of a predefined size and shape is provided, facing an opposite side of a center of said lens; implanting said IOL, said main longitudinal axis B:B positioned on an arbitrary radian; rotating in situ said IOL comprising said plurality of said at least one HCS so that said main longitudinal axis B:B is superimposed on said astigmatism radian A:A; and in situ positioning a capsule peripheral circumferential edge over the groove to fixate the lens at a required angle, prevent rotation of the lens and enable fixation to an anterior capsule.

It is another object of the invention to disclose the method as defined in any of the above, wherein said lens is either a toric lens or a non-toric lens; said at least one haptic being selected from a group consisting of a plate-type haptic, a loop-type haptic and any combination thereof; and said IOL comprises n of said at least one HCS, n being equal to or greater than 2.

It is another object of the invention to disclose the method as defined in any of the above, wherein n is in a range selected from a group consisting of 2 to 4, 6 to 8, and more than 10.

It is another object of the invention to disclose the method as defined in any of the above, wherein said IOL further comprises a member of a group consisting of (i) at least one vertical hole; (ii) at least one horizontal hole; (iii) at least one vertically protruding structure; (iv) at least one horizontally protruding structure, and any combination thereof.

It is another object of the invention to disclose the method as defined in any of the above, wherein said IOL additionally comprises a plurality of clasp-like snaps affixable onto said anterior surface along said main longitudinal axis B:B.

It is another object of the invention to disclose the method as defined in any of the above, wherein at least one of said plurality of clasp-like snaps is selected from a group consisting of a toothed clasp, a clip-like fastener, a clamp-like fastener, a spring fastener, a pliers-like clasp and any combination thereof.

It is yet another object of the invention to disclose a method for fixating an IOL and preferably a toric IOL to an anterior capsule for any indication, including in cases at which the posterior capsule is absent or ruptured, comprising steps of providing an IOL having a main longitudinal axis B:B, an anterior surface of a main plane X:Y and a posterior surface, comprising a lens is interconnected to at least one haptic; affixing at least one vertical capsular stabilizer (VCS) perpendicular to said anterior surface along said main longitudinal axis B:B; when affixed, a fastener head of said VCS protrudes from said anterior surface X:Y so that a gap of a predefined size and shape is provided, facing an opposite side of a center of said lens; if astigmatism correction is required along radian A:A, ex situ affixing a plurality of capsule stabilizing fasteners on said anterior surface along said main longitudinal axis B:B; so that heads of said plurality of fasteners protrude from said anterior surface X:Y so that a gap of a predefined size and shape is provided, facing an opposite side of a center of said lens; implanting said IOL, main longitudinal axis B:B positioned on an arbitrary radian; if required, rotating in situ said IOL comprising said plurality of said at least one VCS so that said main longitudinal axis B:B is superimposed on said astigmatism radian A:A; and, still in situ, positioning a capsule peripheral circumferential edge over the gap to fixate the lens at a required angle, prevent its rotation and enable fixation to the anterior capsule.

It is another object of the invention to disclose the method as defined in any of the above, wherein said lens is either a toric lens or a non-toric lens; and said at least one haptic is selected from a group consisting of a plate-type haptic, a loop-type haptic and any combination thereof.

It is another object of the invention to disclose an IOL as defined in any of the above, wherein said IOL comprises n of said at least one VCS, n being equal to or greater than 2.

It is another object of the invention to disclose an IOL as defined in any of the above, wherein n is in a range selected from a group consisting of 2 to 4, 6 to 8, and more than 10.

It is another object of the invention to disclose the method as defined in any of the above, wherein said IOL further comprises a member of a group consisting of (i) at least one vertical hole; (ii) at least one horizontal hole; (iii) at least one vertically protruding structure; (iv) at least one horizontally protruding structure, and any combination thereof.

It is another object of the invention to disclose the method as defined in any of the above, wherein said at least one VCS is selected from a group consisting of a symmetric clasp, an asymmetric snap, a jeans-button anchor, a hook-containing member, a loop-containing member, a hot-melt snap, a toothed clasp, a spring fastener and any combination thereof.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts, and in which:

FIGS. 1a-d, 2, 3, 4, 5 and 6 schematically illustrate an IOL with added snap-like symmetric and asymmetric hook-like capsular stabilizers according to a first set of embodiments of the invention;

FIG. 7 schematically shows positioning of the capsule peripheral circumferential edge over the capsular stabilizers to fixate the lens at the required angle and prevent its rotation and also enable fixation to the anterior capsule;

FIG. 8 to FIG. 10 schematically illustrate an IOL with button-like capsular stabilizers according to a second set of embodiments of the invention;

FIG. 11 to FIG. 13 schematically illustrate an IOL with pin-like capsular stabilizers according to a third set of embodiments of the invention;

FIG. 14 to FIG. 17 schematically illustrate an IOL with clasp-like capsular stabilizer according to a fourth set of embodiments of the invention;

FIG. 18 to FIG. 31 schematically illustrate an IOL with horizontal stabilizers, modules and a kit thereof, and methods for implanting the capsular stabilizers within the lens structure by the physician according to a fifth set of embodiments of the invention;

FIG. 32 to FIG. 41 schematically illustrate an IOL with vertical capsular stabilizers, modules and a kit thereof, and methods for implanting the capsular stabilizers within the lens structure by the physician according to a sixth set of embodiments of the invention;

FIGS. 42a-b schematically illustrate a cross section of the human eye with an IOL in a typical orientation with the posterior capsule intact, according to an embodiment of the invention;

FIGS. 42c-d schematically illustrate a cross section of the human eye with an IOL in a typical orientation in the case of a missing posterior capsule, according to an embodiment of the invention;

FIGS. 43a-c schematically illustrate a cross section of the human eye with an IOL in a typical orientation with the posterior capsule intact, according to yet another embodiment of the invention; and

FIGS. 44a-b schematically illustrate two preferable Capsulorhexis asymmetric openings that mark the exact positioning angle required to angularly position the IOL, such Capsulorhexis openings will be preferably performed by a computerized laser system or technologies that create the Capsular opening using a pre-shaped heating element or alike technology, according to a few embodiments of the invention: FIG. 44a depicts a technique of Capsulorhexis with notches angularly positioned according to the required positioning angle of the IOL and preferably a toric IOL and FIG. 44b depicts a technique of ellipse Capsulorhexis, with the long axis of the ellipse positioned according to the required positioning angle of the IOL and preferably a toric IOL;

FIGS. 45-47 schematically illustrates a cross section of an IOL implanted in a human eye and capsular stabilizers according to a few embodiments of the invention;

FIGS. 48a and 48b schematically illustrates a set of capsular stabilizers 451 incorporated in a plate-type haptic and a loop-type haptic, accordingly;

FIGS. 49a-d schematically depict four different capsular stabilizers according to a few embodiments of the invention;

FIG. 50, schematically showing three steps in a method of implanting capsular stabilizers-containing haptic; and

FIGS. 51a-b and 52, schematically disclosing tools, modules, a medical device, a kit and methods for implanting capsular-stabilizers containing IOL according to a few embodiments of the invention.

### DETAILED DESCRIPTION OF THE PREFERRED EMBODIMENTS

It is to be understood that the figures and descriptions of the present invention have been simplified to illustrate elements that are relevant for a clear understanding of the present invention, while eliminating, for the purpose of clarity, many other elements found in typical lenses, lens systems and methods. Those of ordinary skill in the pertinent arts may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the pertinent arts.

The terms “haptics” refers hereinafter to a portion which is connected to the IOL optic and functions to support, fixate and center the optic within the capsule in a predefined orientation.

The term ‘intraocular lens’ (IOL) refers hereinafter to an artificial implantable lens, positioned by a capsulotomy fixation within a capsular bag of the eye or sutured or anchored in cases where the bag is not present or is ruptured, to replace the focusing power of a natural lens that has been surgically removed, typically during cataract surgery. In many cases, the IOL is a pseudophakia member, e.g., an open loop intraocular lens, a closed loop intraocular lens, or a plate intraocular lens, which substitutes for a natural lens. Usually IOLs are made of materials such as silicone, hydrophilic acrylate (hydrogel), hydrophobic acrylate, polymethyl methacrylate materials, etc. An IOL implant is configured for the eye as per an appropriate prescription to provide effective vision for a user.

Fixation of IOL's to the anterior capsule using the capsular stabilizers may be done in cases that require predetermined angular positioning, e.g., astigmatism, where toric IOLs are required. The terms “toric optic”, “toric” and “toric IOL” will be used interchangeably to refer to an optic configured to correct astigmatism, and may include monofocal, multifocal, aspheric, non-aspheric, toric, and EDOF lenses.

The terms “hole”, “bore” and “aperture” interchangeably refer to an opening in a matter.

It is also in the scope of the invention when the term related to treating astigmatism is further related to the treatment and correction of myopia and presbyopia and other medical indications associated with refractive errors.

The term “capsular stabilizer” refers hereinafter to at least one fastener provided useful, when assembled, for preventing postoperative rotation of an IOL or for fixating the IOL to the anterior capsule peripheral circumference.

The term ‘about’ refers herein after to any value within 25% of the defined measure.

Fixation of an IOL to the anterior capsule using the capsular stabilizers may be also done in cases that do not require predetermined angular positioning. It is hence in the scope of the invention wherein any IOL may be fixated to the anterior capsule by the capsular stabilizers, typically in medical situations where the bag's posterior part is either ruptured, torn or missing.

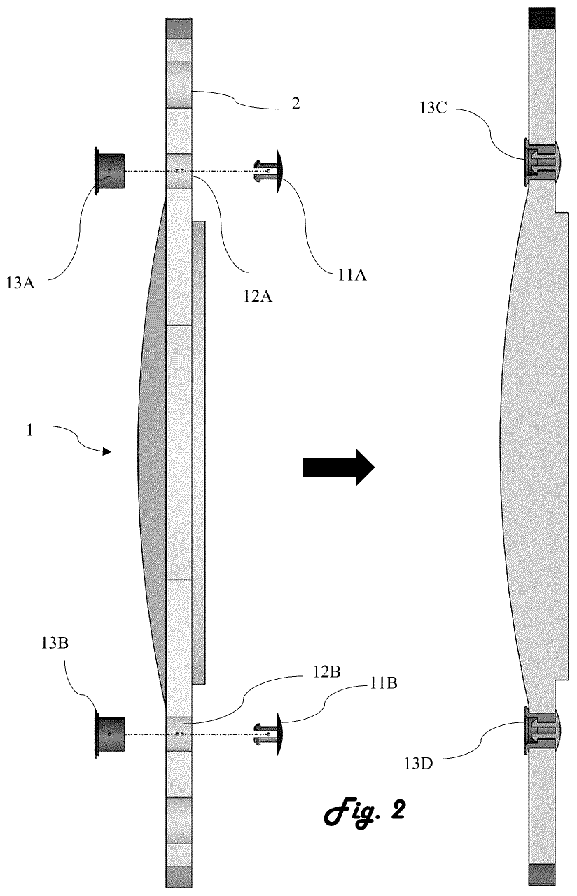

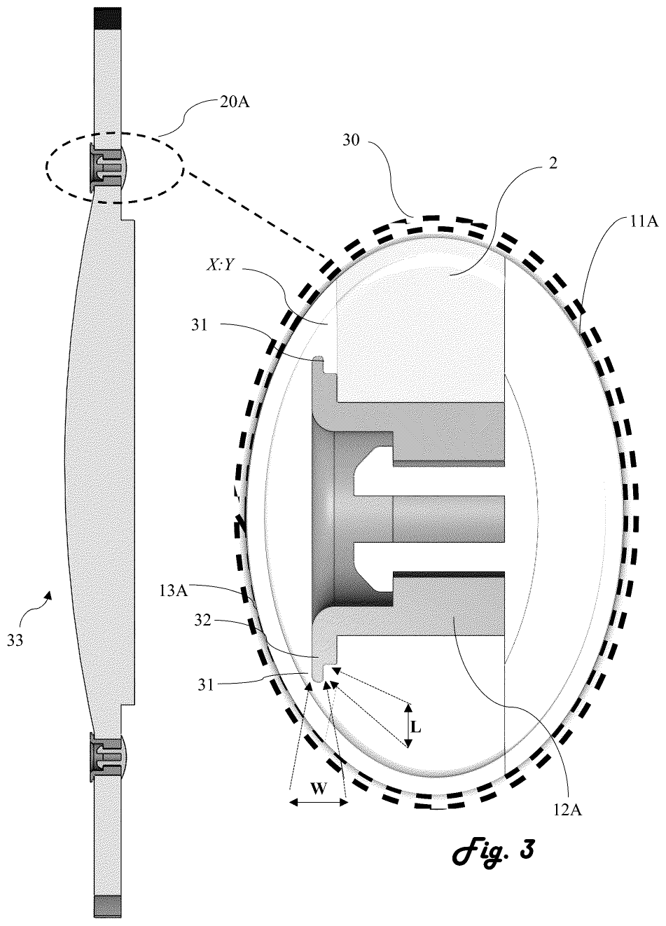

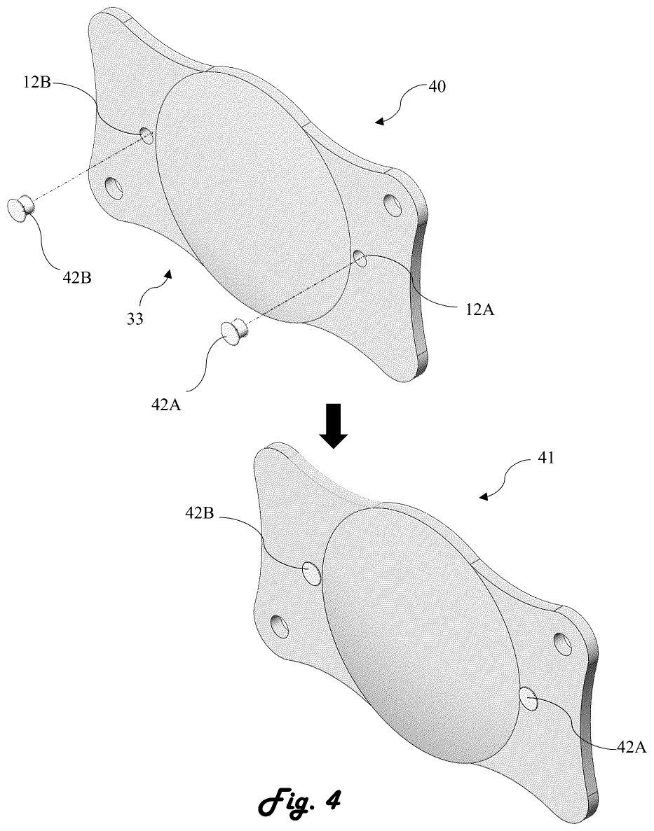

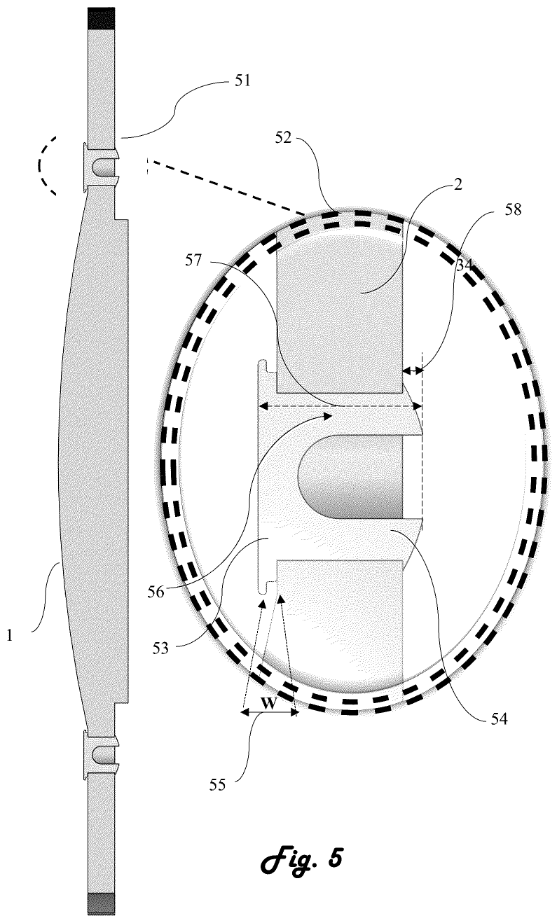

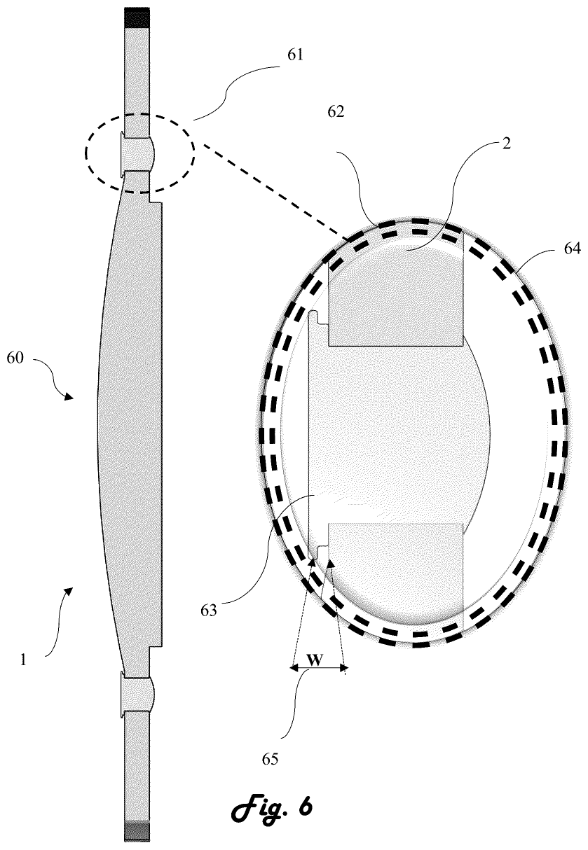

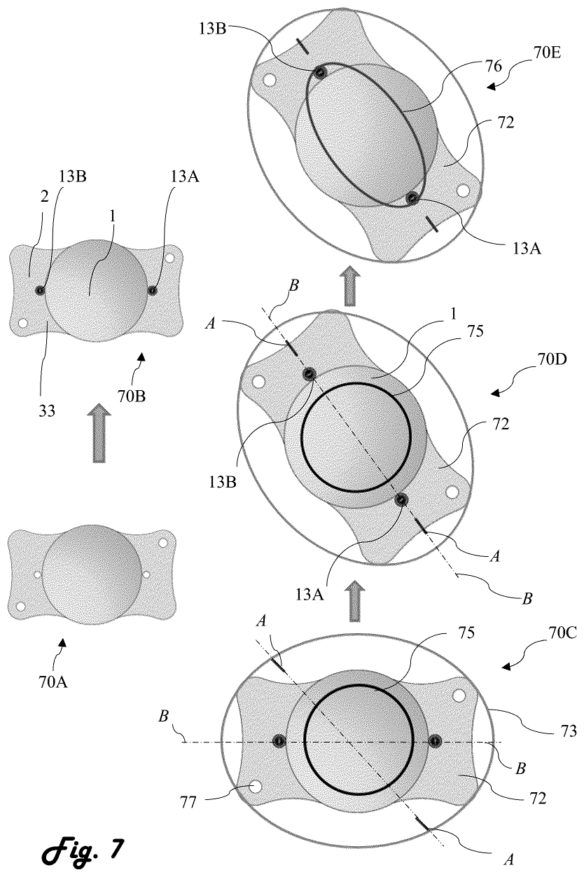

According to one embodiment of the invention, in the case of a toric IOL having a main longitudinal axis B:B, as shown in FIG. 7, an anterior surface of a main plane X:Y and a posterior surface, comprising a lens having a center and interconnected to at least one haptic, a plurality of fasteners is reversibly affixable on the anterior surface along the axis B:B. When affixed, the heads of the capsular stabilizers protrude from the anterior surface X:Y so that a gap of a predefined size and shape is provided, facing an opposite side of a center of said lens. It is according to yet another embodiment wherein the capsular stabilizers are attached to an IOL vertically, horizontally or both. It is according to yet another embodiment wherein the capsular stabilizers are characterized by a member of a group consisting of, inter alia, a knob-like shape, a rivet-like shape, a pin-like shape, a screw-like shape, a coil-like shape, a stud-like shape, a snap-like shape; a jeans-button anchor, a click-in stud, a male-female two part arrangement, a toothed-clasp, a spring-clasp, with the capsular stabilizer placed in either a vertical or a horizontal orientation relative to plane X:Y, and any combination or derivative thereof.

The capsule stabilizers may be positioned in at least one of (i) holes created during the manufacturing process, and (ii) holes made post-manufacturing by the physician on the sterile table, using a dedicated or a non-dedicated puncturing tool (not shown). The holes can be made in the IOL circumference either vertically or horizontally.

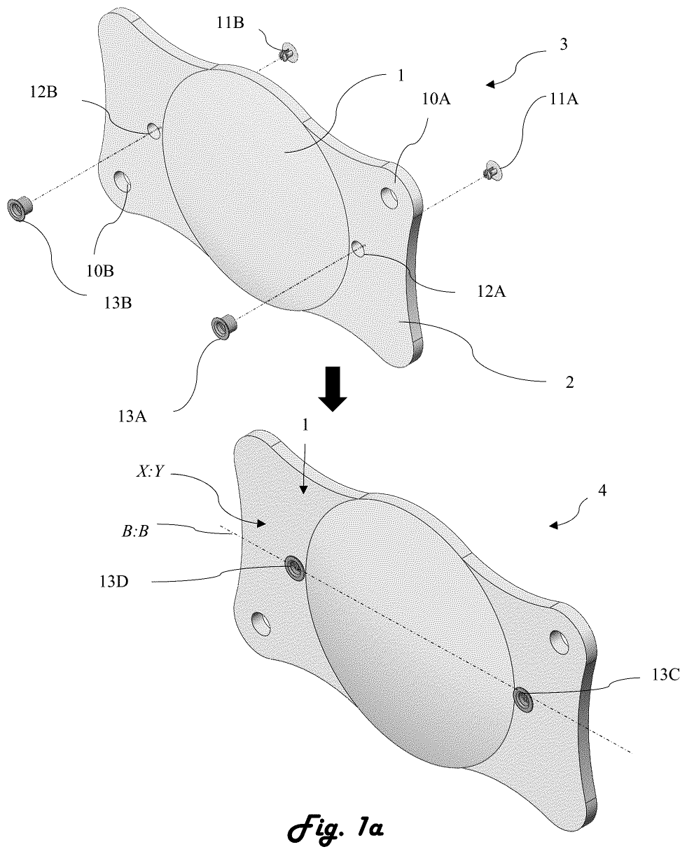

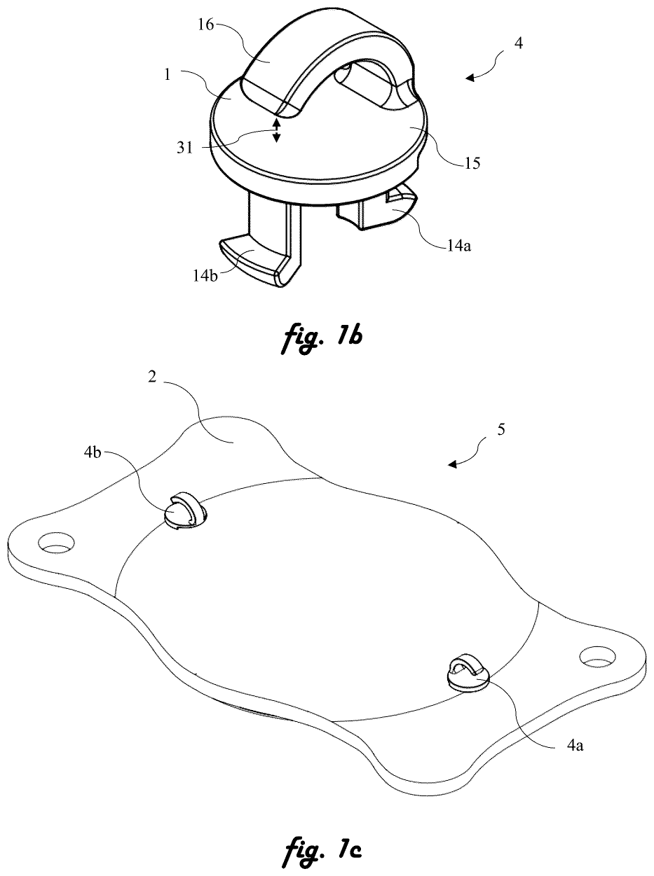

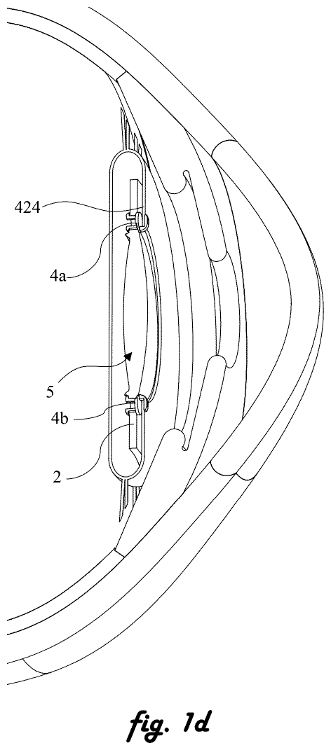

Reference is now made to FIGS. 1a-d and 2. FIG. 1a schematically illustrates a first exploded perspective view 3 of a lens with a set of two capsular stabilizers (11A, 13A, and 11B, 13B), according to an embodiment of the invention. The capsular stabilizers are positioned within holes adjacent to the IOL. The holes are either made during the production of the lens or made on site with a puncturing tool (not shown). The anterior surface (1) is a surface having a main plane X:Y and main longitudinal axis B:B. A second perspective view is presented at the bottom of the page, depicting an integrated scheme of the same (4). FIG. 2 schematically illustrates a side cross sections of FIG. 1a. In an embodiment of the invention a swivel-able IOL-haptic member comprises inter alia an IOL connected with at least one side-structure, (haptics, 2).

According to another embodiment of the invention, the IOL is characterized by an obviously convex anterior surface (1) and a relatively flat posterior surface, with a radius of curvature of the anterior surface always being smaller than the radius of curvature of the posterior surface.

A first haptic comprises at least one first hole (or bore) 12A and a second haptic, located at the other side of the lens, comprises at least one second hole (or bore) 12B. At least one first female capsular stabilizing fastener 13A, characterized by symmetric properties, is insertable within bore 12A and at least one first male snap capsular stabilizing fastener 11A is insertable within bore 12A to securely connect (see 13C) to the at least one first female snap capsular stabilizing fastener 13A. Similarly, at least one second female capsular stabilizing fastener 13B, similarly symmetric, is insertable within bore 12B and at least one second male snap capsular stabilizing fastener 11B is insertable within bore 12B to securely connect (see 13D) to the at least one second female snap capsular stabilizing fastener 13B.

According to one set of embodiments of the invention where, on haptics, or in a location provided adjacent to the interconnection of haptic with IOL or on a periphery of the optic, a plurality of positioning bores may be provided useful for reversibly or temporarily affixing the capsular stabilizers. Further apertures (e.g., 10A and 10B) and structures (not shown) may be also located on the IOL or haptics thereof.

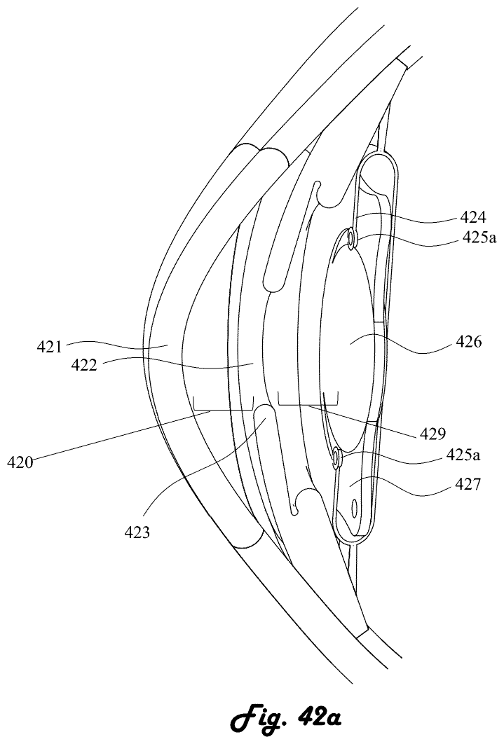

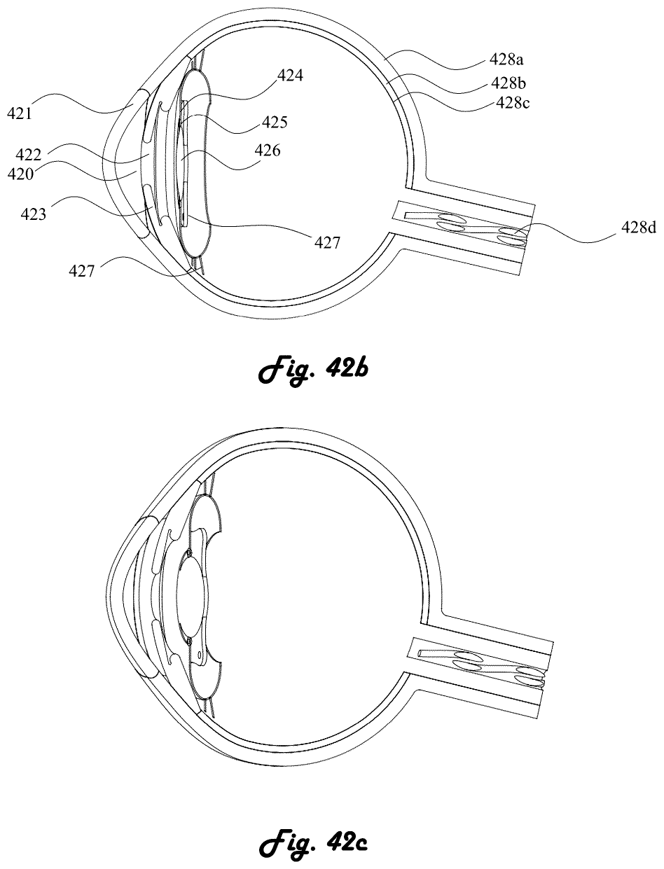

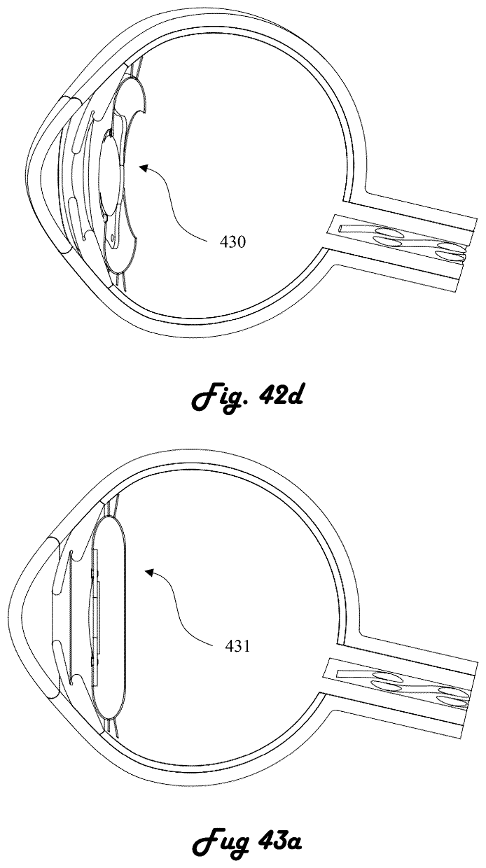

Reference is now made to FIGS. 1b to 1d, schematically illustrating asymmetric male snap capsular stabilizing fastener 4, comprising at the anterior side at least one peg, catch or hook-like member 16 forming a gap 31 of a predefined size and shape, e.g., width and length between the hook end and haptic surface 15 (for a side view, see e.g., FIG. 3). Hook-like member 16 is positioned and configured to hold the peripheral circumferential edges of the capsule 424, see also e.g., FIGS. 1d and 42a-b. The posterior side of capsular stabilizer 4 comprises inter alia one or two more legs, see 14a and 14b. FIG. 1c shows the IOL's (5) anterior side, of a plate-type (2) with attached asymmetric capsular stabilizers 4a and 4b. FIG. 1d schematically depicts a cross section of an eye, showing both posterior and anterior portions of an IOL 5, when it is implanted within an intact posterior capsule.

Reference is now made to FIG. 3, schematically illustrating a lateral cross-section of an IOL 33 according to an embodiment of the invention. FIG. 3 further illustrates a zoomed-in view (30) of the capsular stabilizing fastened snap 20A. The haptic (2) incorporates a hole 12A through which from the anterior side a stepped-head snap 13A is inserted, followed by the insertion from the posterior side of a curved-head (smooth) snap 11A, snaps 13A and 11A fasten to each other thus lock in position. The stepped head 13A on the anterior surface of plane X:Y forms a gap 31 of predefined size and shape, e.g., width (W), length (L) between snap's rim 32 and haptics' surface X:Y.

In an exemplary manner provided herein in a non-limiting manner, W ranges between about 50 μm to about 150 μm, whilst L ranges between about 200 μm to about 1,000 μm, where “about” refers to a value within 20% of the defined value.

Reference is now made to FIG. 4, schematically illustrating perspective views of IOL 33 and capsular stabilizers 42A and 42B according to another embodiment of the invention. In view 40, click-in capsular stabilizing studs 42A-42B are inserted to bores 12A-12B, respectively; to yield an integrated IOL implant 41, namely IOL 33 with a set of two stabilizers (42A, 42B). FIG. 5 illustrates the same in a set of lateral cross sections, where zoomed-in view 52 shows the plugged capsular stabilizing stud 51. The click-in stud comprises a smooth-leg stopper 54 facing the posterior surface, and a stepped head 53 facing the anterior surface (1) providing, when assembled, a gap of a predefined size and shape (55). Capsular stabilizers 42A and 42B click in position and fixate in bores 12A-12B. Flexibility of the capsular stabilizers' necks' 56 dimensions, the IOL's material or both enable posterior side protrusion 58 to pass through the bore (see neck's length 57) and fixate on the posterior plane, fixating the capsular stabilizers in place

Reference is now made to FIG. 6, schematically illustrating lateral cross-sections of IOL 60 with capsular stabilizers 61 according to another embodiment of the invention. Here, a rivet-like capsule stabilizing knob 61 (see also zoomed-in view 62), optionally a heat-forming member produced and assembled e.g., during production or on site, is inserted into a bore of haptic 2. Smooth leg 64 is provided on the posterior surface while a stepped head 63 of a predefined size and shape (65) is provided on anterior surface.

The IOL is implanted into the capsular bag of the eye. A standard approach for performing such surgery is to open the anterior part of the crystalline lens capsule by Capsulorhexis, which ensures a circular opening through which the lens matter is removed and through which the IOL is inserted. The capsule is opened by various techniques know in the art, such as by tearing, cutting, burning by use of a laser and any combination thereof. The preferred placement of the IOL is in-the-bag. The lens matter is often removed using phacoemulsification which uses ultrasound to disintegrate and aspirate the lens matter through a small incision; alternatively, the lens matter may be disintegrated manually or using a femto-second laser. Once the lens matter has been removed, the IOL is implanted through the opening in the anterior capsule and placed in the empty bag.

Reference is hence made to FIG. 7, schematically illustrating a means and method of positioning and rotationally anchoring IOL 33 with capsular stabilizers (13A, 13B). Following insertion of the IOL into the bag through the anterior opening (Capsulorhexis), the IOL is rotated by the physician to the required angle, then the peripherals of the bag's opening are positioned over the capsular stabilizers as shown. Anterior surface 1 of IOL 75 is characterized a first curvature and a second curvature (both are not shown). For example, the first curvature may be along a first meridian and oriented vertically, and the second curvature may be along a second meridian and oriented horizontally. The IOL can either include capsular stabilizers that were either positioned or plastic-injected during the production process or alternatively added by the physician on site. For example, preparation of an IOL with capsular stabilizers may be done in the operating room on the sterile table; in cases in which the IOL does not include holes/bores a puncturing dedicated tool may be added.

Steps of preparing a plate-haptic IOL (70A) and stepped-head snaps, e.g., capsule stabilizers 13A-13B can be as follows: the surgeon opens a sterilized capsular stabilizer 70A from its sterile envelope. Then (70B), the surgeon opens an IOL sterile envelope or blister, all being provided remote from the patient, and the surgeon attaches the capsular stabilizers (e.g., 13A, 13B) to the corresponding holes. In cases in which the IOL does not include holes, a dedicated puncturing tool can be added to the kit and used accordingly. Now the IOL, comprising the capsular stabilizer 33 is ready to be implanted in situ.

The lens with plate-haptics 72 are positioned in an arbitrary orientation, namely with the stabilizer's main longitudinal axis B:B and interconnecting snaps 13A and 13B are not superimposed on the predefined astigmatism radian (axis A:A). In a further step, see view 70D, the surgeon rotates the IOL (e.g., clockwise, to 3 clock) so that the IOL axis B:B is superimposed on astigmatism radian A:A. In a further step, see view 70E, the surgeon gently pulls the bag's peripheral circumference and positions it within gaps of the stepped head 13A and 13B, hence fixating the IOL to avoid postoperative rotation of the capsular stabilizer.

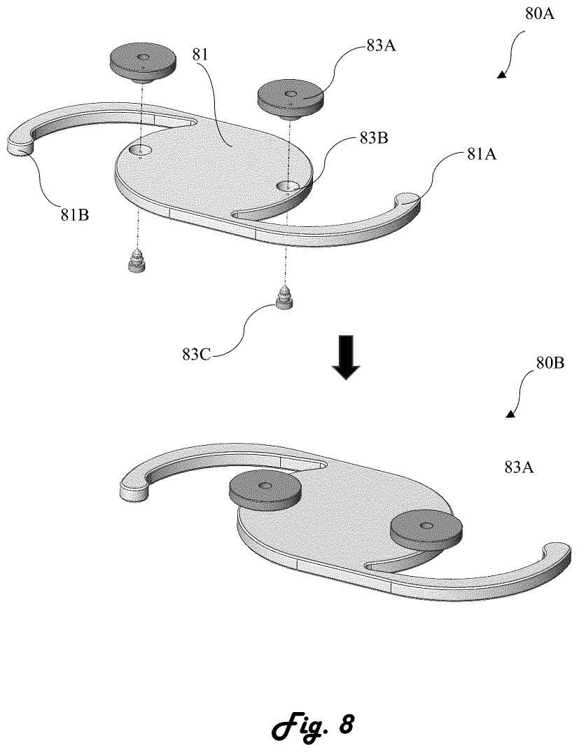

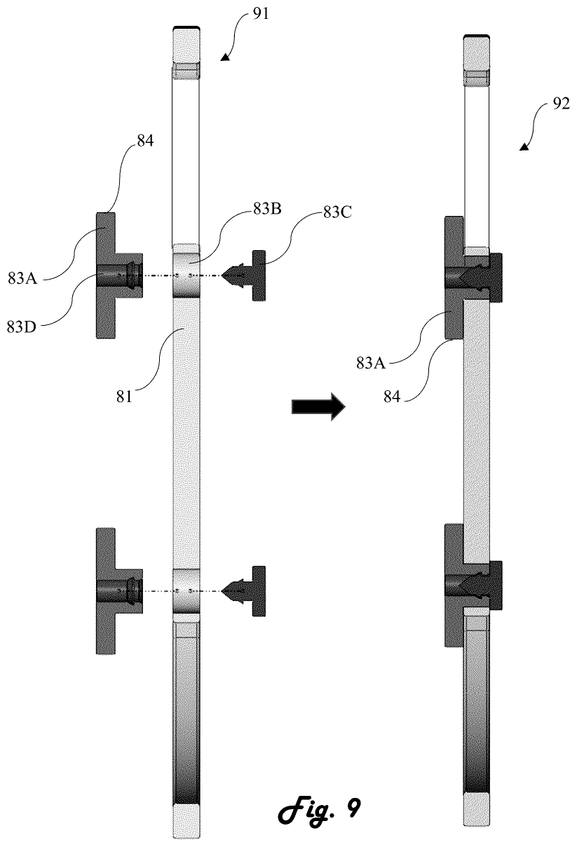

Reference is now made to FIGS. 8 and 10, schematically illustrating a loop-haptic IOL according to another embodiment of the present invention, a haptic (81) comprising novel vertical capsule stabilizing studs, here e.g., jeans-button like anchors 83A. In FIG. 8, views 80A and 80B depict an exploded perspective view and an integrated view of the same, respectively, where a female head 83A is insertable within bore 83B, and fastened by a male stud 83C. Loop haptics 81A and 81B are shown. FIG. 9 schematically illustrates a lateral cross-section of the same. Views 91 and 92 are an exploded scheme and an integrated scheme of the same. FIG. 10 schematically illustrates a zoomed-in view 101 of a cross-section of the same, depicting the male-female interconnection of the stabilizer's head 83A and the stud 83C. Horizontal protrusion 84 juts horizontally to create a connecting notch for the peripheral circumference of the capsular bag. In an exemplary manner, provided herein in a non-limiting manner, the anterior vertical dimension W ranges from about 100 μm to about 300 μm whilst posterior protrusion gap should be minimal. Here again, capsular stabilizers are positioned within holes adjacent to the IOL. The holes are either made during the production of the lens or made on site with a dedicated puncturing tool (not shown).

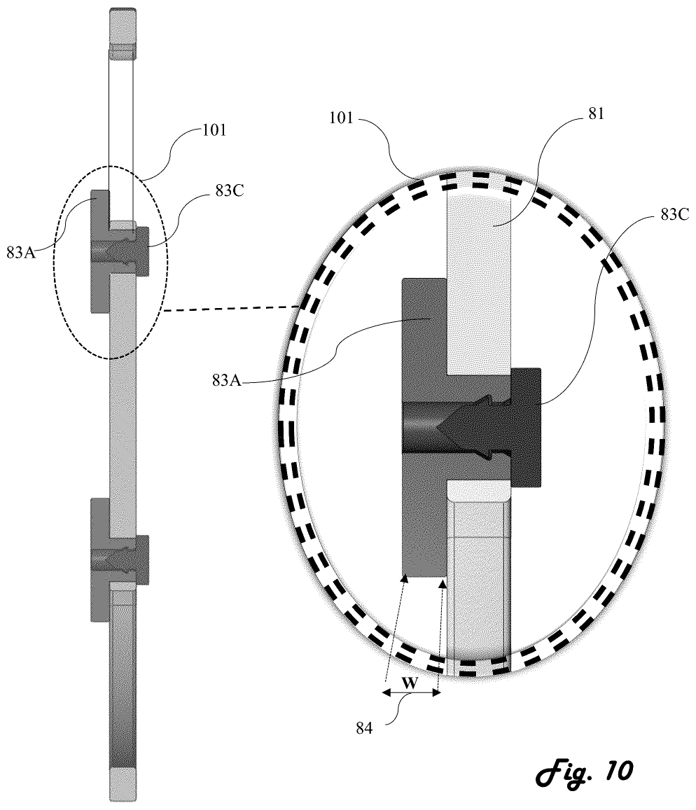

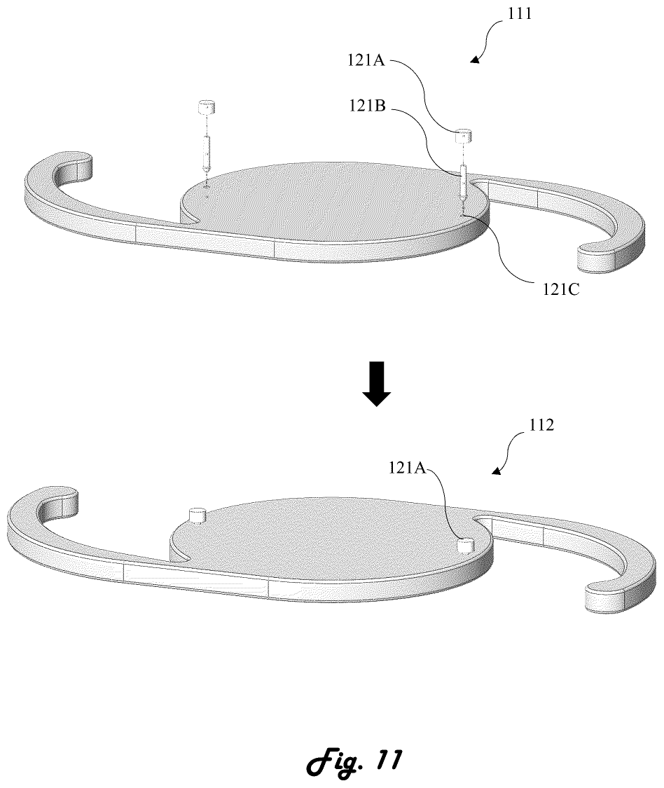

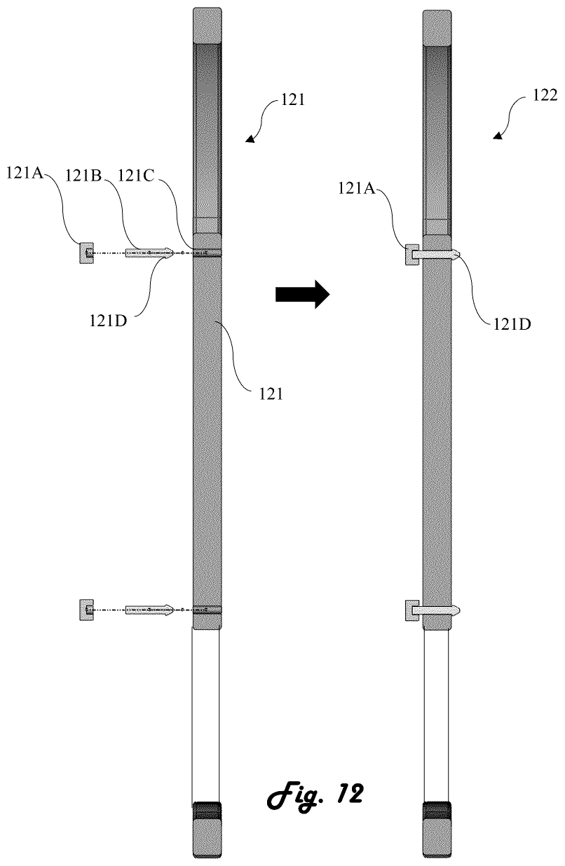

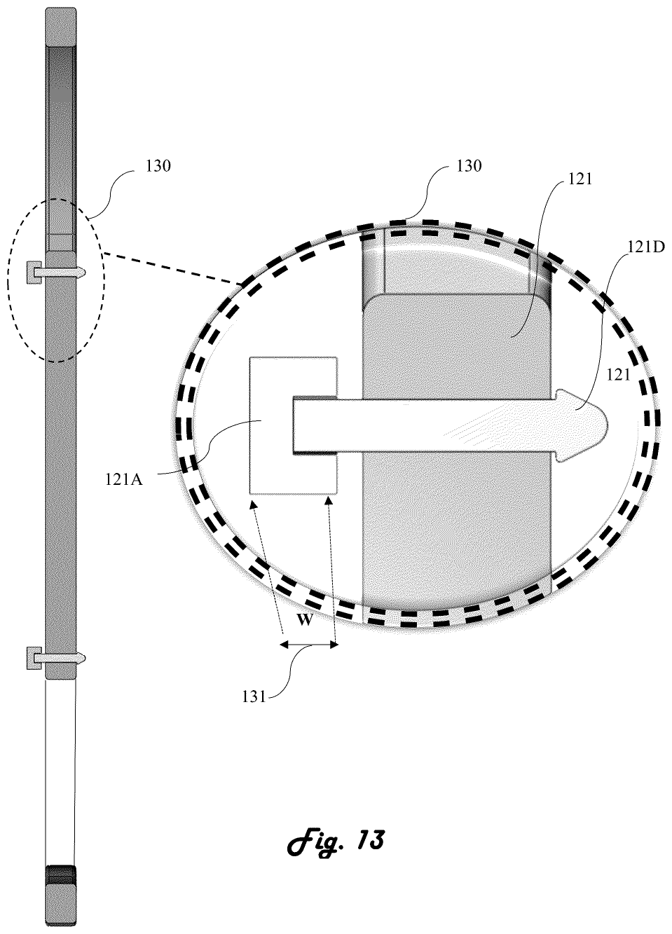

Reference is now made to FIG. 11, schematically illustrating a loop-haptic IOL according to another embodiment of the present invention, comprising a set of novel vertical capsule stabilizing studs, here e.g., vertical pins. Views 111 and 112 show an exploded perspective scheme and an integrated scheme of the same, respectively, where a head 121A interconnected with a pin 121B is insertable within bore/hole 121C. FIG. 12 schematically illustrates a lateral cross-section of the same. Schemes 121 and 122 are an exploded view and an integrated view of the same, respectively. The bulb head of 121D secures the pin in its position in the posterior surface. FIG. 13 schematically illustrates a zoomed-in view 130 of a cross-section of the same, depicting head 121A and stud 121D. Head 121A protrudes to wide W (131) vertically from the anterior surface of the IOL, thus creating a slot, notch or groove between head 121A and the anterior surface of the IOL to allow positioning of the peripheral circumference of the capsular bag. As the above, capsular stabilizers are positioned within holes adjacent to the IOL and holes are either made during the production of the lens or made on site with a dedicated puncturing tool (not shown).

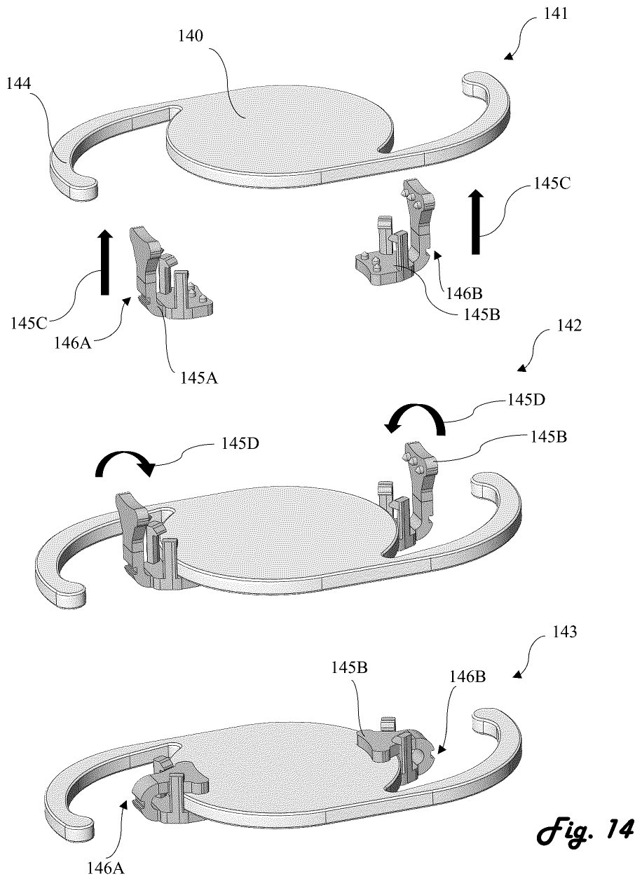

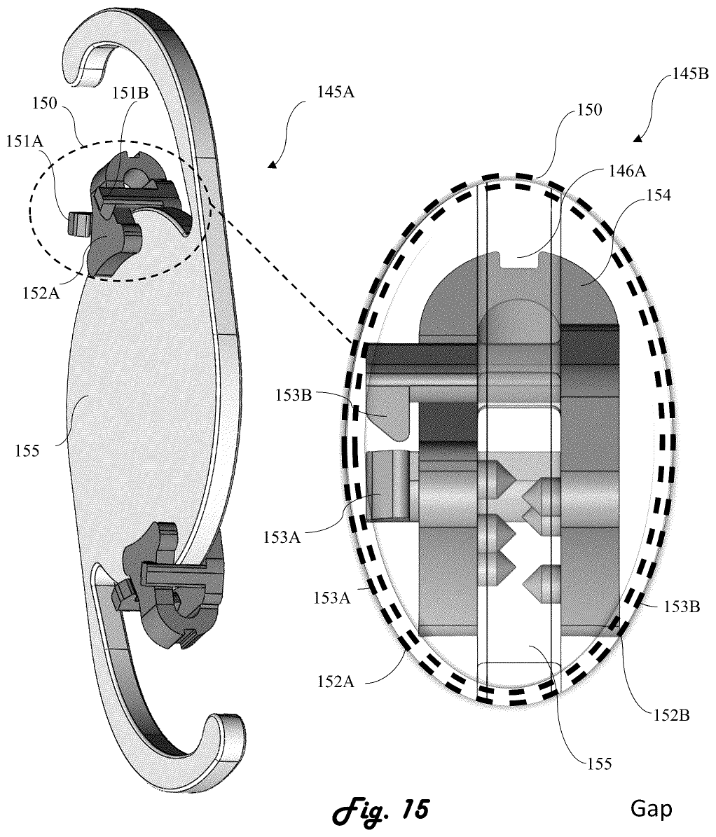

Reference is now made to FIG. 14, schematically illustrating a loop-haptic IOL that does not require holes for attaching the fasteners according to another embodiment of the present invention, comprising novel vertical capsule stabilizing studs, here e.g., single piece clasp-like snaps 145A and 145B. Each snap, e.g., on its curved neck, comprises a groove of a predefined size and shape (146A and 146B). Schemes 141, 142 and 143 show perspective views of the same, where a grooved snap 145A is provided ex situ adjacent (145C) to one portion of the IOL 140, near a first haptic, and, similarly, a grooved snap 145B is provided adjacent (145C) to an opposite portion of the IOL 140, near a second haptic. The Clamps (145D) may be added to the IOL during production or alternatively the surgeon adds the clamps at the surgery room by clamping the snaps from their open configuration to their closed configuration, hence affixing them to the IOL body (view 143). Snaps 145B and 145A have a groove of a predefined size and shape (146) for positioning the peripheral circumference of the anterior capsule. FIG. 15 schematically illustrates a perspective view on the left (145A) and a zoomed-in lateral cross-section of the same on the right (145B). Zoomed-in view 150 schematically illustrates a cross-section of the same, depicting the stepped head portions 152A, 152B, a plurality of teeth (153A, 153B) located on both sides of the clamped stabilizer (154). The teeth penetrate the body of the IOL, thus further securing it to a defined location and orientation. At the anterior surface of the IOL, a plurality of studs (151A, 151B) are configured to lock the clamp in its closed configuration. Groove 146A is of a predefined size and shape provided useful for accommodating the peripheral circumference of the anterior capsule in a predefined position.

Reference is now made to FIG. 16, schematically illustrating a loop-haptic IOL according to another embodiment of the present invention, comprising novel vertical capsule-stabilizing studs, here e.g., a toothed clamp 145. Schemes 161 and 162 show perspective views of the same, with a toothed clamp 145A provided ex situ adjacent (154C) to one portion of the IOL's anterior portion 155, near the first haptic and vice versa for the second toothed clamp. By this, the surgeon fastens (154D) the stabilizer from its open configuration to a closed configuration, and hence affixes the clamps to the IOL's rim (view 162). Grooves 146A and 146B are also shown. FIG. 17 schematically illustrates a perspective view on the left, and a zoomed-in lateral cross-section of the same on the right (171). Zoomed-in view 171 depicts stepped head portions 152A, 152B on the anterior side of the implant, a plurality of teeth (153A, 153B) located on both sides of the clamping capsule stabilizer (154). The teeth are configured to penetrate, attach or otherwise pierce the body of the IOL or haptic thereof, thus further securing the clamp in its place. Bridge 154 is a spring-member configured to forcefully shut the two jaws 152A and 152B, thus ensuring that the teeth 153A and 153B firmly grip both sides of the IOL 155. Groove 146A, of a predefined size and shape (146), is also illustrated.

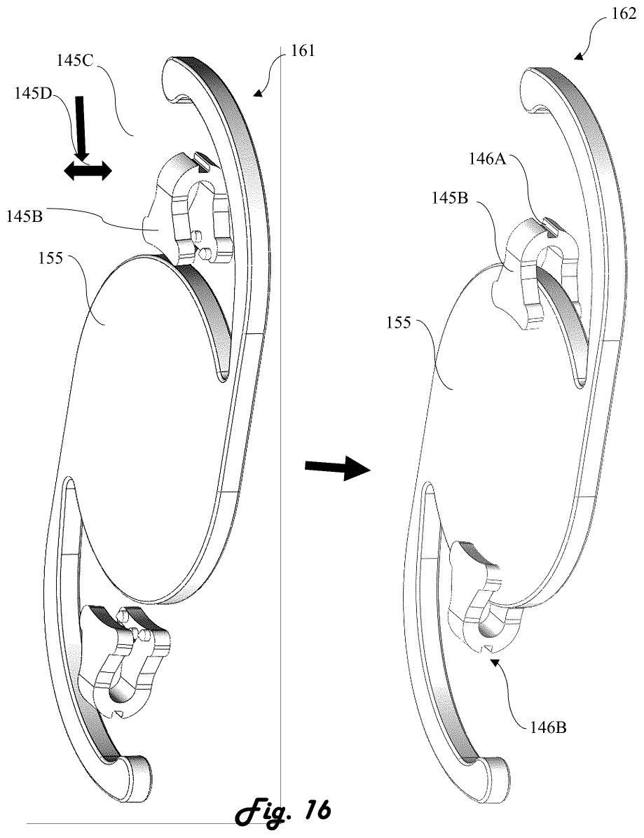

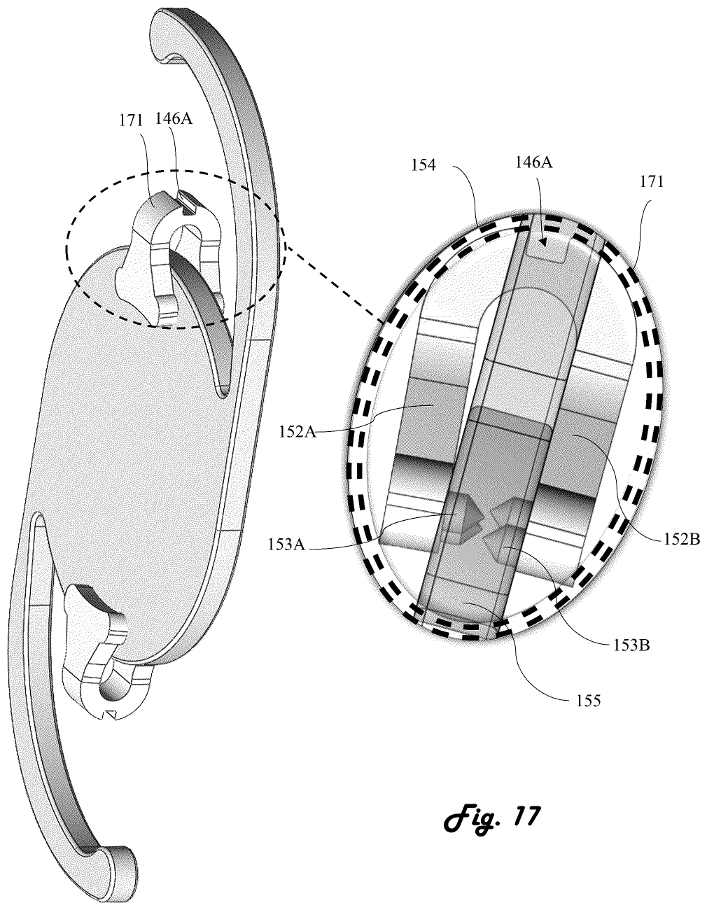

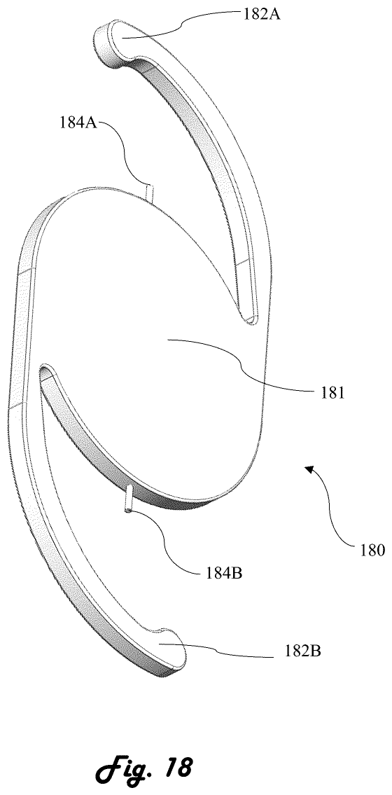

Reference is now made to FIG. 18, schematically illustrating a loop-haptic (182A, 182B) IOL (181, view 180) according to another embodiment of the present invention. It comprises novel horizontal capsule stabilizing studs, here e.g., pin-like stabilizers 184A and 184B. It is well in the scope of the invention where the aforesaid pin-like stabilizer is provided as at least one protruding member of any 3D orientation, shape, dimensions and materials.

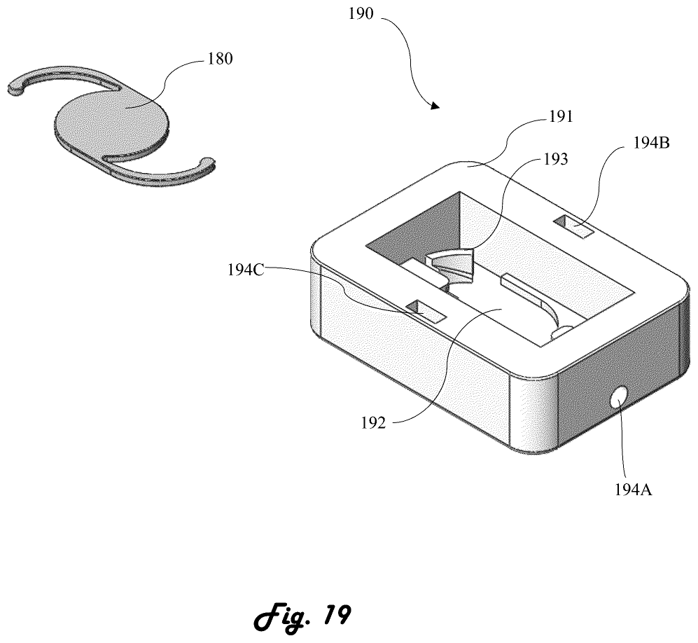

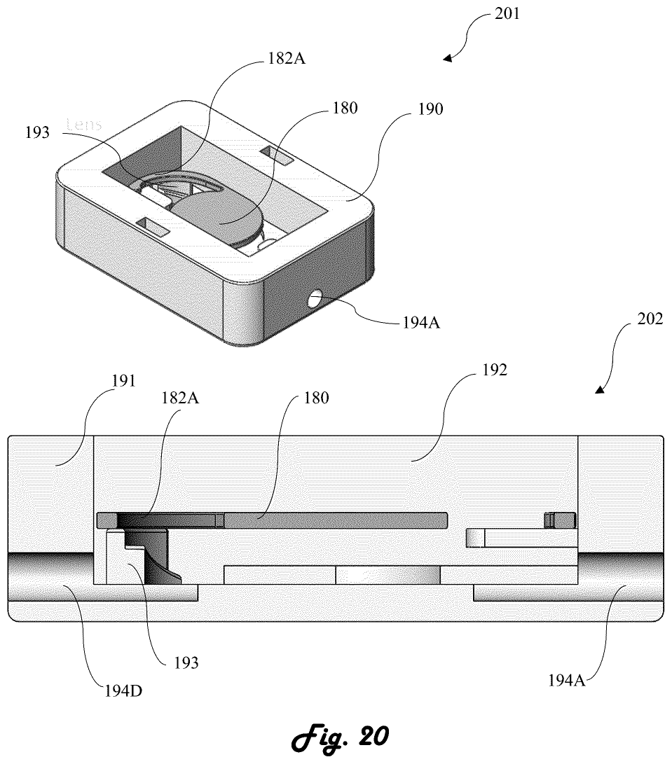

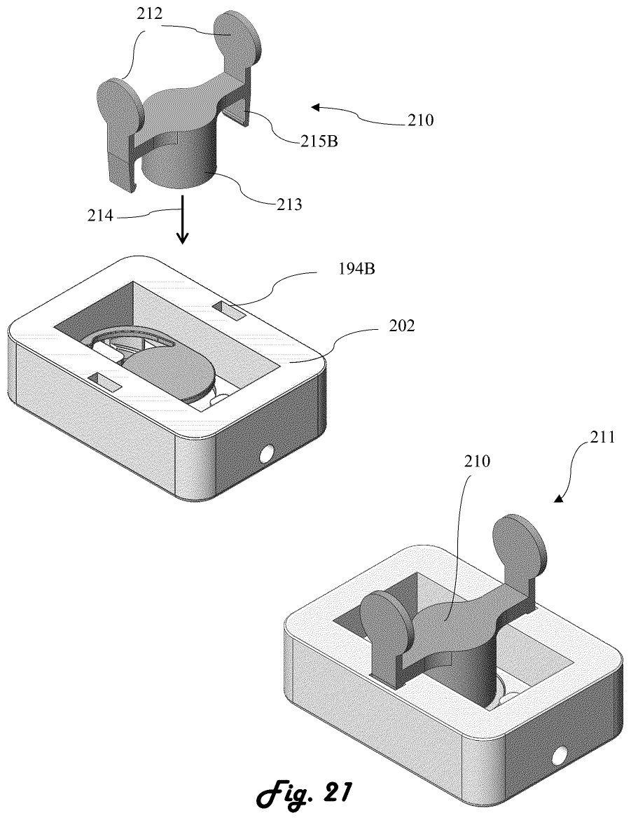

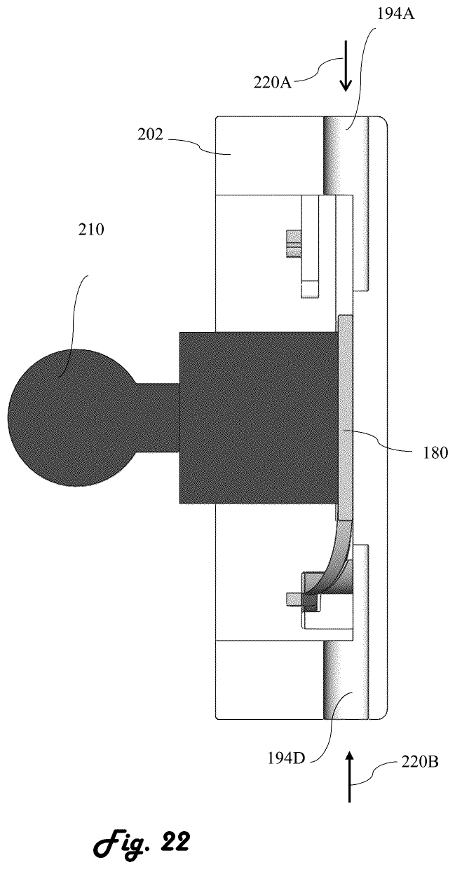

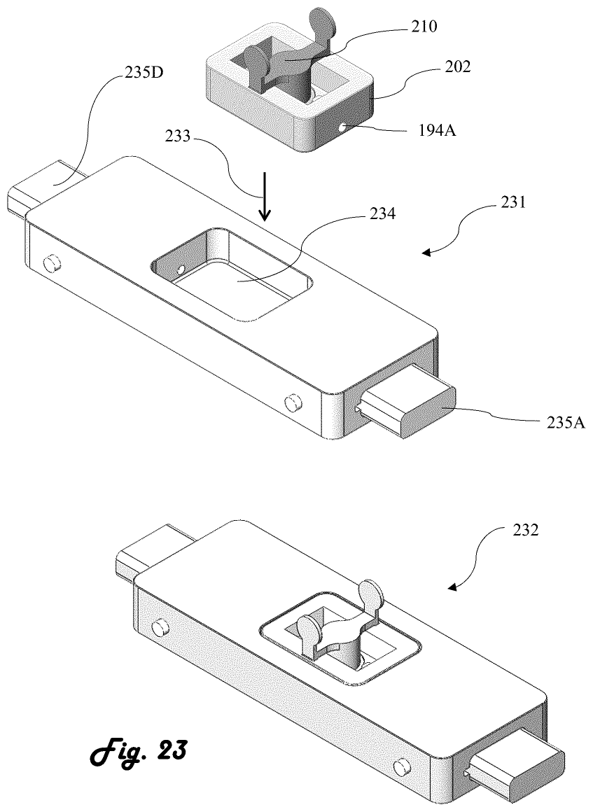

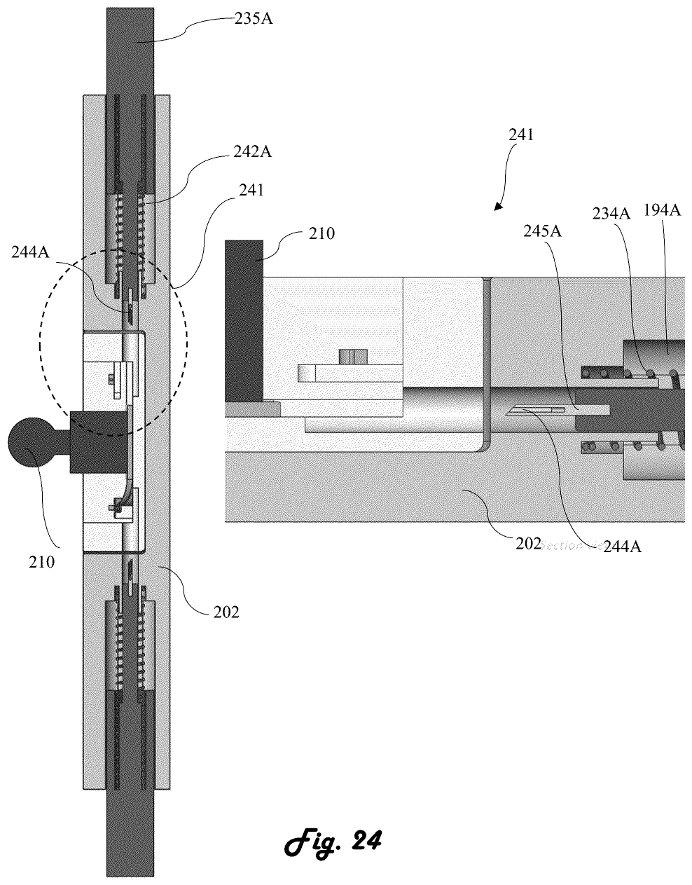

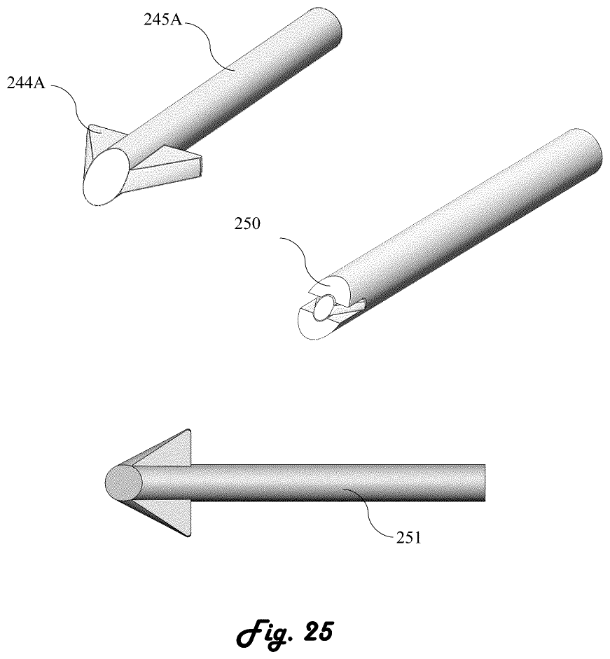

Reference is now made to FIGS. 19-31, schematically illustrating means and methods for the assembly of the horizontal pin-like capsular stabilizer according to another set of embodiments of the invention. In FIG. 19, a pin-less common IOL 180 is inserted within housing 190 of an adaptor designed to hold the specific IOL outlines; characterized by e.g., walls 191 that define a cavity 192 having inside haptic-accepting jigs 193 and a plurality of apertures 194A-194C (aperture 194D is not shown). It is in the scope of the invention wherein a kit includes various adapters for a variety of lens outlines. Reference is now made to FIG. 20 schematically illustrating (see perspective view 201) an IOL 180 placed within adapter 190, where haptic 182A rests in jig 193. View 202 is a lateral cross section of the same, schematically illustrating walls 191 and cavity 192, in which IOL 180 is placed, and haptic 182A rests in jig 193. Apertures 194A and 194D are further shown. In FIG. 21, perspective views are shown. A lens positioner 210 is schematically illustrated. It comprises a rounded body 213, two tongs 212 and locks (215B). The lens positioner clicks into the adaptor as shown below, see view 211. Locks (215B) are inserted within the corresponding apertures (194B) so that a secure assembly (211) is provided. Reference is now made to FIG. 22, schematically illustrating a cross section of the secure assembly (211). The lens positioner presses the IOL downwards while the haptic distal ends are supported and accordingly remain lifted to enable a route for insertion of the anchors. This section view shows a lens positioner 210 which presses the IOL 180, and two insertion (220A and 220B) routes (194A and 194D, respectively). In FIG. 23, perspective views are shown. In this step, the loaded adaptor is positioned in its dedicated slot within the insertion platform. View 231 schematically illustrates the loaded adaptor (202-210) snugly inserted 233 in slot 234 of an insertion platform, which comprises a button (see e.g., 235A) which fits aperture 194A, and an opposite button 235D which fits aperture 194D (not shown here). View 232 schematically illustrates the aforesaid assembly. Refence is now made to FIG. 24, schematically illustrating a lateral cross section of the same on the left side, and a zoomed-in view (241) on the right. These illustrations show a horizontal pin-insertion mechanism according to an embodiment of the invention. Button 235A module fits within aperture 194A located in housing wall 202 so that a needle 245A is enabled with a reciprocated linear movement within the aperture. The needle is actuated by means of a button 235A and spring 234A. In the end of the pin, a spike 244A is provided. Refence is now made to FIG. 25, schematically illustrating a set of perspective views of an anchor having a capsule stem 245A which is incorporated with directional spikes 244A to prevent retraction of the stabilizing pin from the lens when the needles are retracted. Loaded needle 250 and a side view of the anchor are also presented. It is in the scope of the invention wherein the design of the stem, spikes, anchor, pins and needles is different.

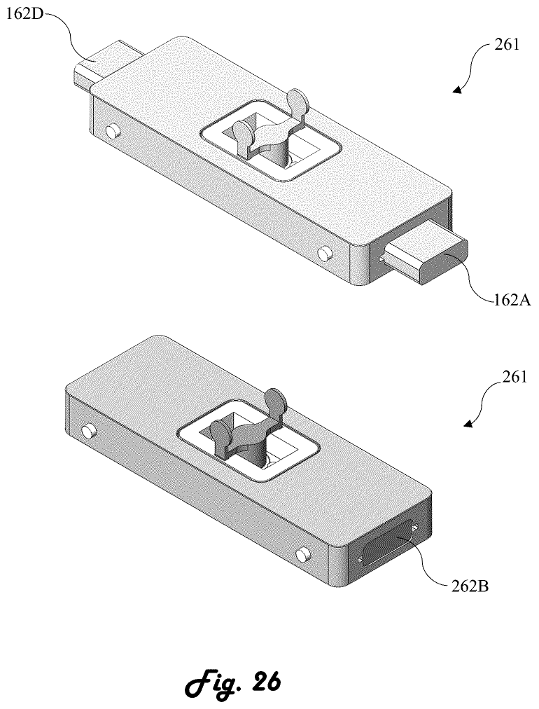

Reference is now made to FIG. 26. The operation mechanism, according to an embodiment of the invention, comprises various steps, e.g., those that follow: The insertion platform incorporates sliding needles that are factory preloaded with anchors of a desired design. As shown in views 261 and 262, the surgeon presses the insertion platform's buttons 162A and 162D to simultaneously activate the needles. The loaded needles then penetrate the lens circumference and retract due to the incorporated retraction springs. The preloaded anchors remain in a horizontal configuration within the lens while slightly protruding from the lens circumference. It is in the scope of the invention that a mechanism can be provided to ensure that both needles and/or anchors are inserted and retracted simultaneously, such as adding toothed racks and a reciprocating gear to mechanically link the movement of the linear mechanisms, not shown. FIG. 27 schematically illustrates a lateral cross-section in which loaded needles 245A and 244A penetrate to the lens. FIG. 28 schematically illustrates a perspective view 261, a lateral cross-section 281 and a zoomed-in cross section 282. When the needles are released and retracted in a linear movement, the anchors 244A are left in a predefined horizontal position 181 within the lens circumference.

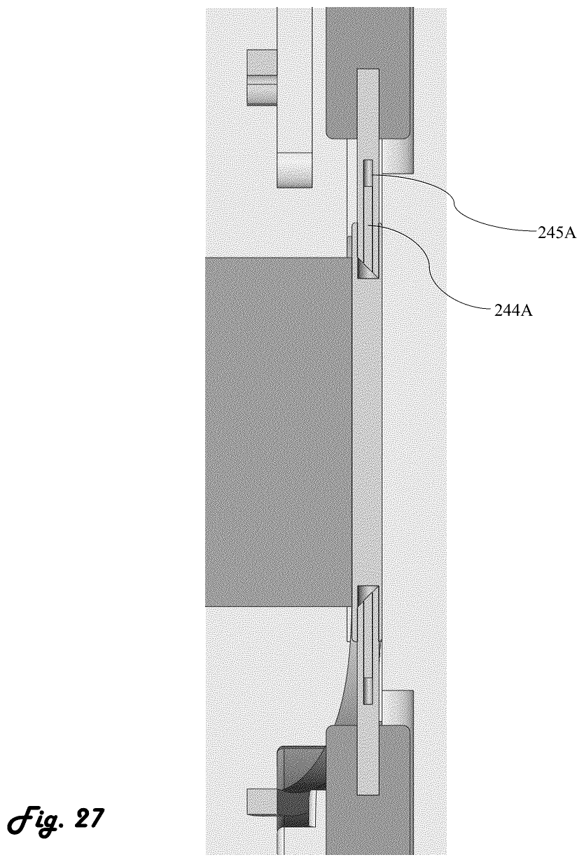

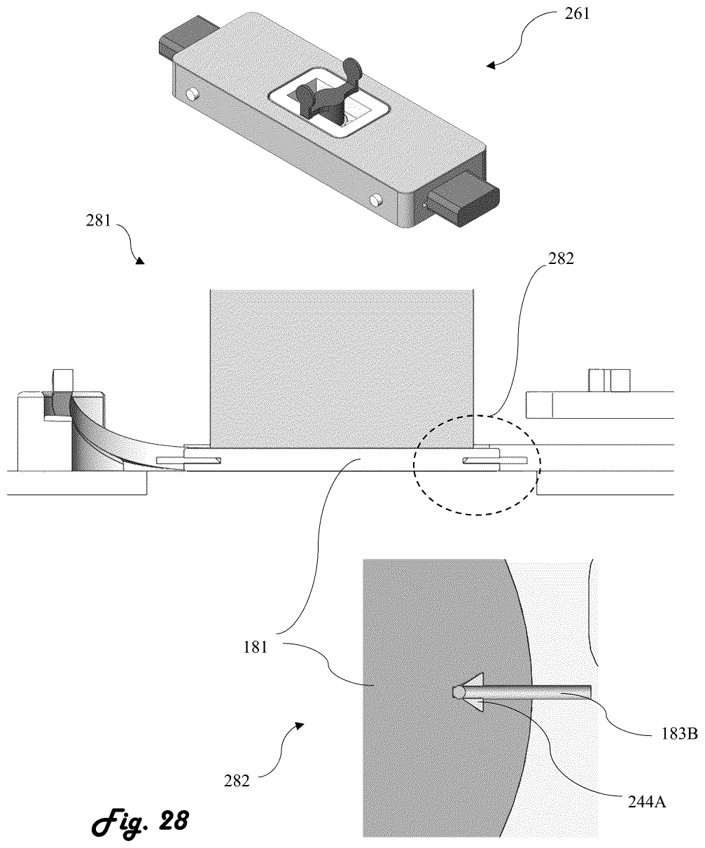

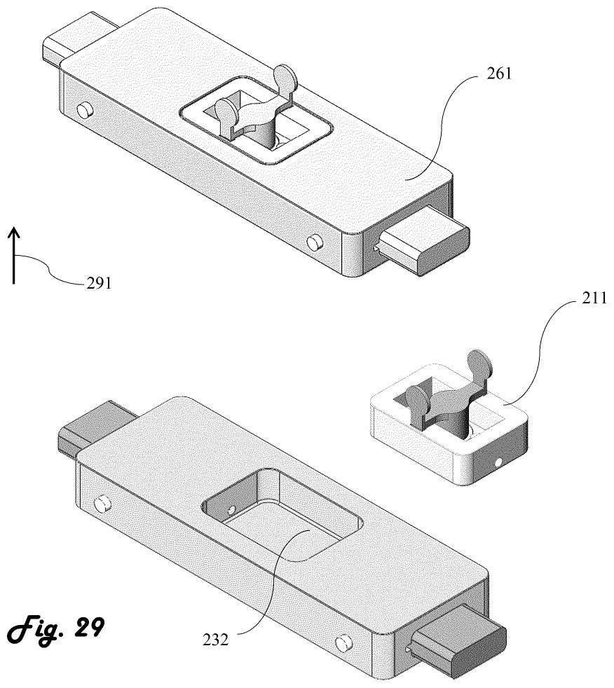

The disassembly process of device 261 is simple and intuitive. Reference is now made to FIG. 29. Buttons are released simultaneously and the adaptor 211 is removed 291 from the insertion platform's slot 232. Then, as shown in FIG. 30, lens positioner 210 is removed from the adaptor 202, so that lens 180 is accessible. In a further step, as shown in FIG. 31, lens 180 is taken out 311 from the adaptor 194.

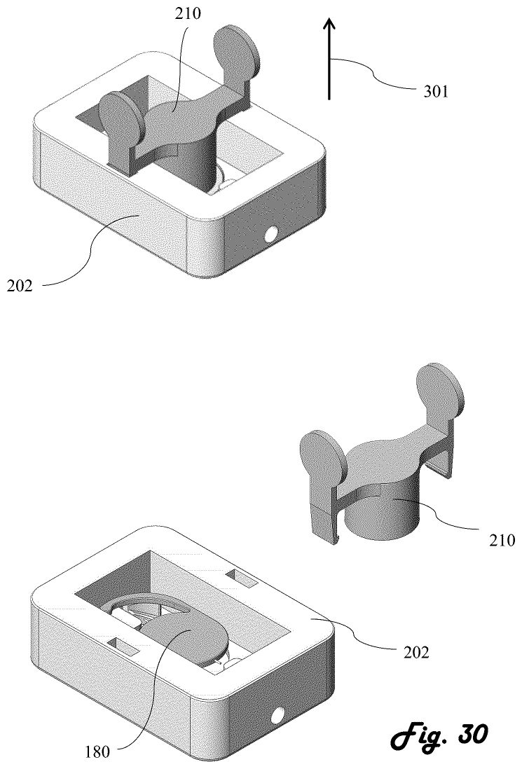

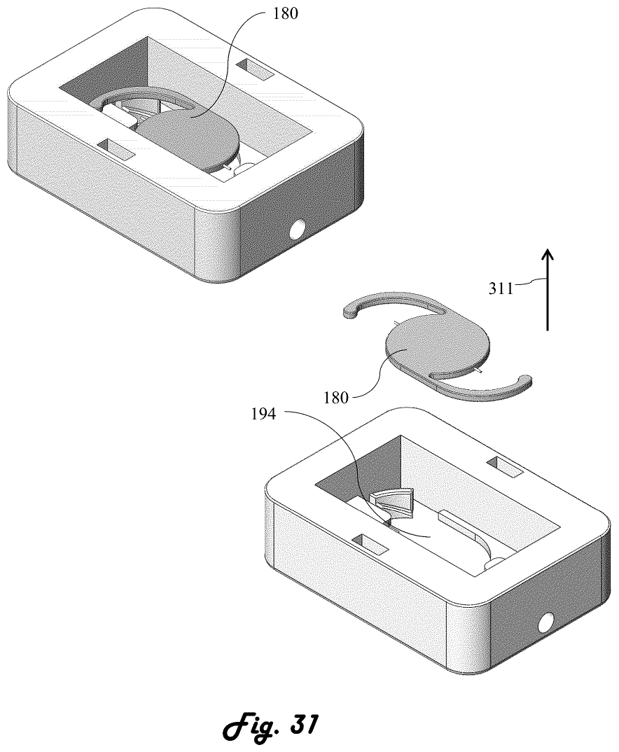

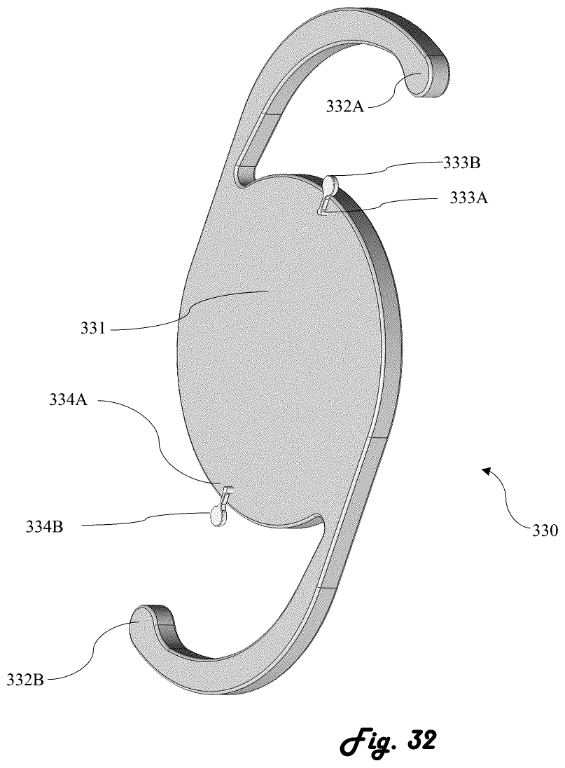

Reference is now made to FIG. 32, schematically illustrating a perspective view of an IOL 331 containing vertically inserted and horizontally jutting capsular stabilizing pins 334B and 333B according to yet another embodiment of the invention. It comprises, inter alia, two haptics 332A and 332B, and an IOL 331. When assembled, it further comprises at least one and possibly several vertically inserted and horizontally jutting capsule stabilizing pins, e.g., stems (333A, 334A) comprising pins or rounded heads (333B, 334B, respectively). It is well within the scope of the invention where the aforesaid pin-like member is provided as at least one protruding member of any shape, orientation, dimensions and materials.

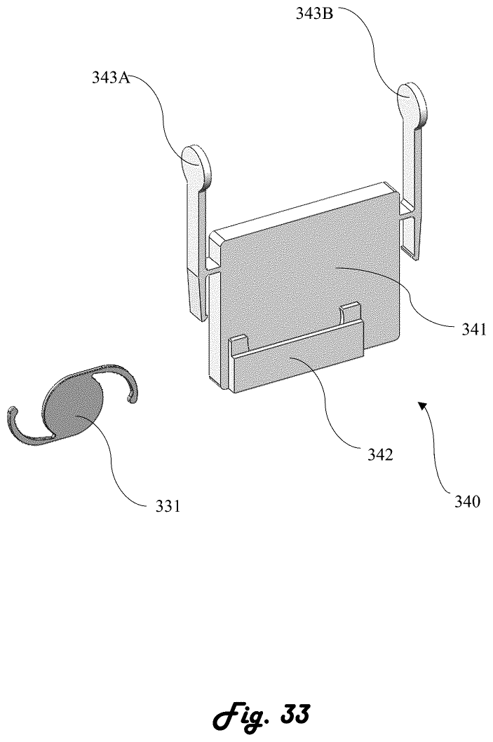

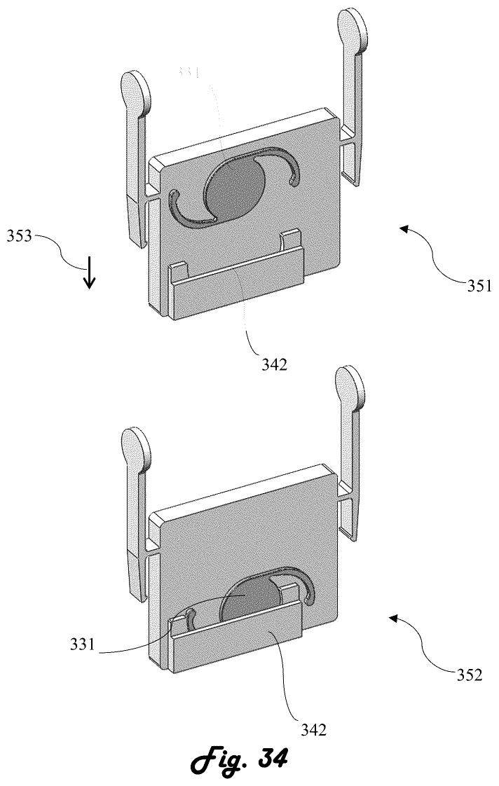

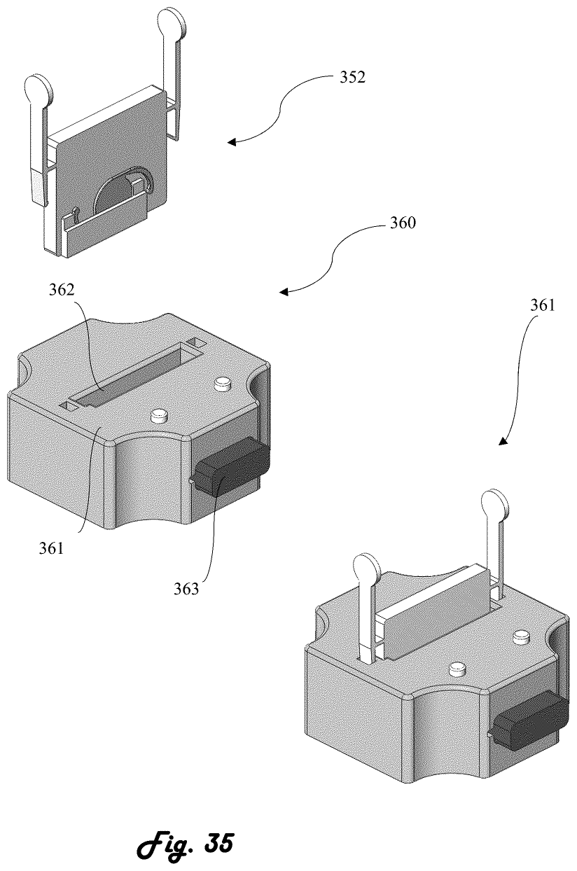

Reference is now made to FIGS. 33 to 41, schematically illustrating means and methods for the assembly of the vertically inserted and horizontally jutting capsular stabilizing pins to the IOL according to another set of embodiments of the invention. In FIG. 33, a pin-less IOL 331 is inserted into a slot 342 in the housing 341 of a matching adaptor (340) designed to hold that particular pin-less IOL. The adaptor is characterized by e.g., two tongs 343A and 343B, a body 341 and a pocket-like slot 342, into which a pin-less capsular stabilizer 331 can be inserted. In a first stage, provided ex situ, as depicted in FIG. 34, the surgeon slides 353 the IOL 331 within pocket 342, see views 351 and 352. It is well in the scope of the invention that a kit may comprise various adapters for a variety of lens outlines, as a specific lens may fit a specific adaptor. In FIG. 35, as shown in perspective views 360 and 361, the loaded adaptor 352 is positioned in its dedicated slot 362 within an insertion platform 361 which comprises two buttons, see, e.g., 363.

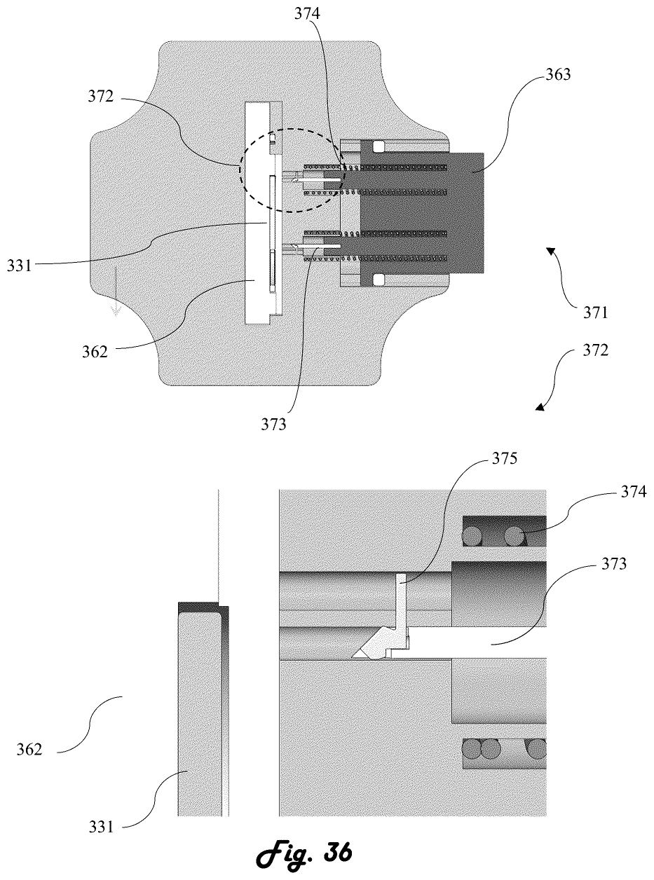

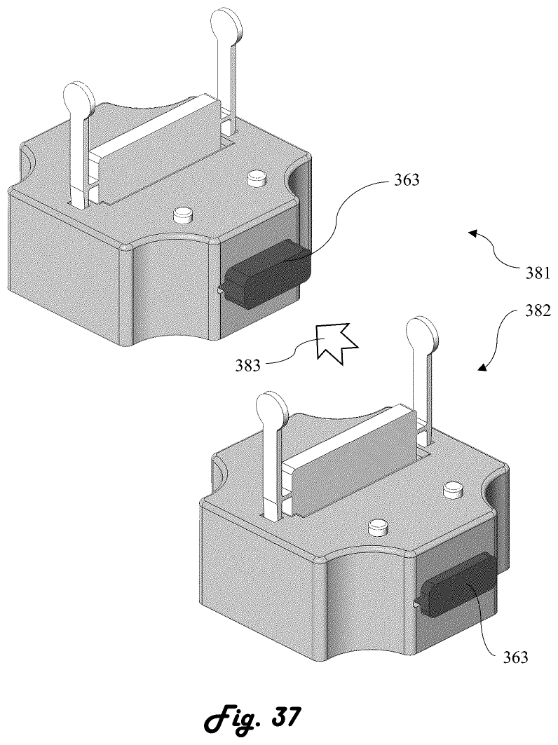

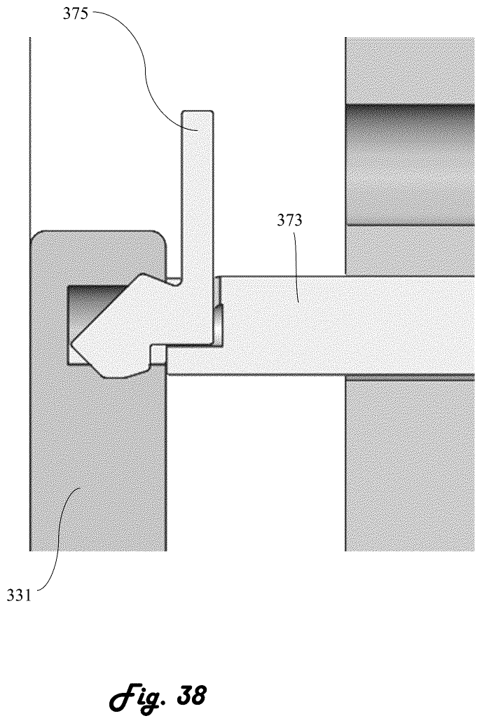

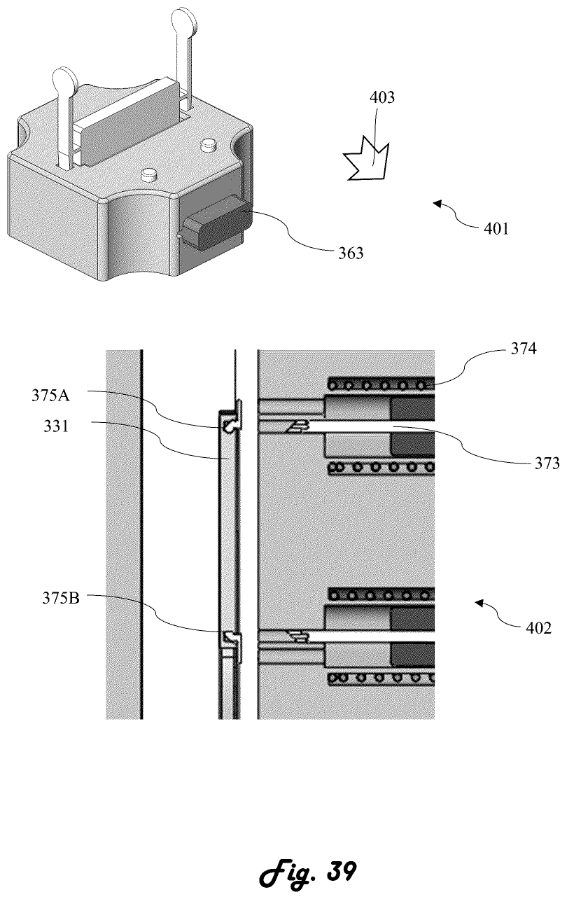

Reference is now made to FIGS. 36 and 39. FIG. 36 schematically illustrates two lateral cross sections, 371 and a zoomed-in view 372 of the same. The operation of the device comprises a few steps: when insertion platform 361 is introduced, it incorporates sliding needles 374 that are factory preloaded with the desired anchors 375A and 375B. Then, the surgeon presses (383, see FIG. 37, perspective views 381 and 382) the insertion platform's buttons 363 to simultaneously activate the needles 373. The loaded needles then penetrate the lens 331 vertically (see 375 and 373 in cross section of FIG. 38) and retract due to retraction springs 374 incorporated there. The preloaded anchors 375 remain within the lens 331, positioning the outer flaps that serve as capsular stabilizers. FIG. 39 schematically shows that, when the needle is released 403 (see perspective view 401), it retracts, leaving the vertically positioned and horizontally jutting capsular stabilizers, see cross section view 402.

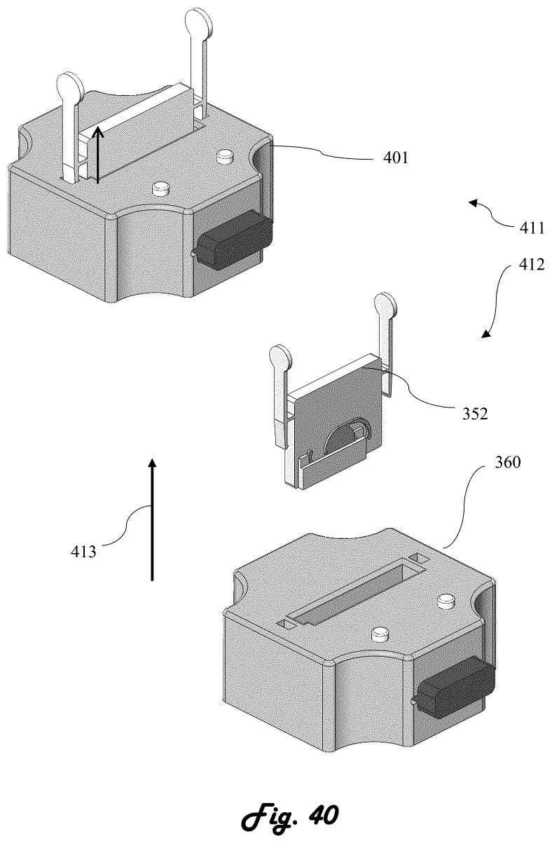

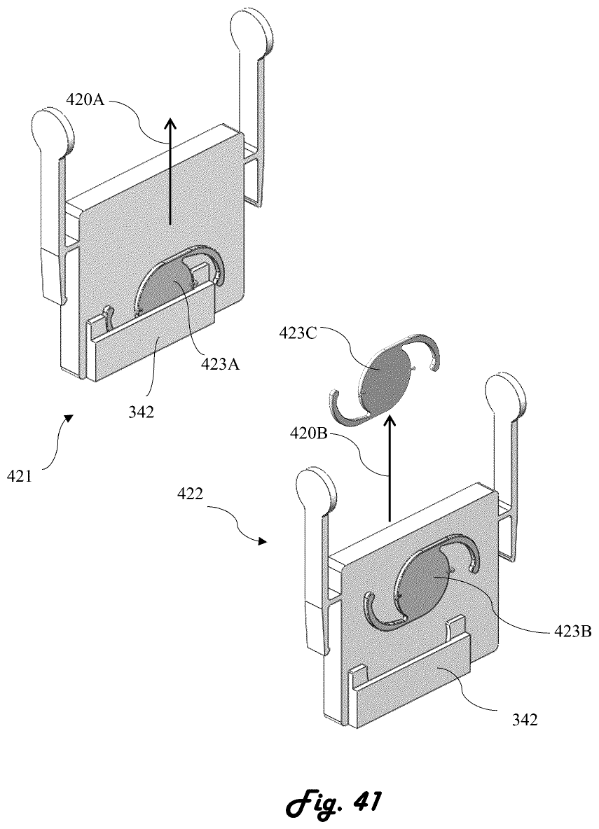

Here again, the disassembly process of device 401 is simple and intuitive to operate, see FIGS. 40 and 41. Reference is made to FIG. 40. The button is released and the adaptor 352 is removed 413 from the insertion platform's slot 360, see perspective views 411 and 412. Then, as shown in FIG. 41, see perspective views 421 and 422, lens 423A slides out (420A) of pocket 342, then it 423B can be taken away (420B), now incorporating the capsular stabilizers (423C).

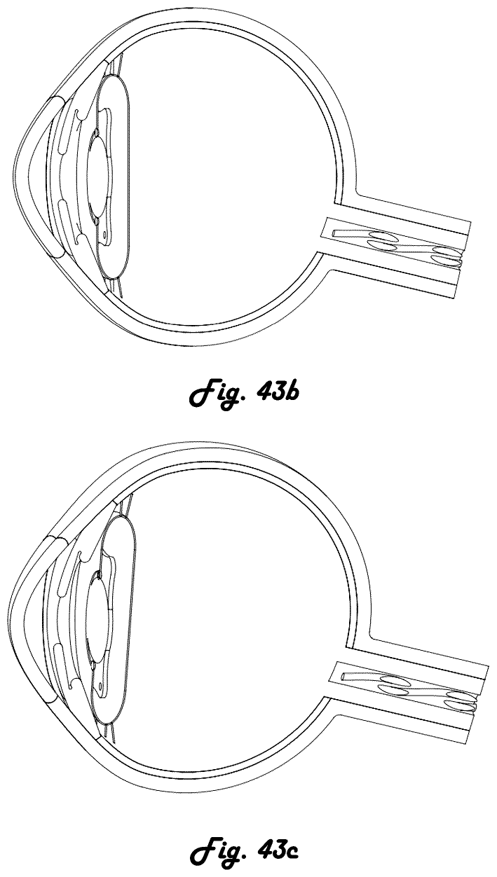

Reference is now made to FIGS. 42a-d and 43a-c schematically illustrating various orientations of a cross-section of an IOL implanted in a human eye, according to two embodiments of the invention. FIG. 42a depicts a zoomed-in view of the anterior cavity: anterior chamber 420 and posterior chamber 429. In the anterior chamber illustrated, e.g., the cornea (421), iris and pupil thereof (422), and ciliary body (423) are shown. FIG. 42b depicts a zoomed-out view of the same, where e.g., the sclera (428a), choroid (428b), retina (428c) and optic nerve (428d) are shown. An implanted IOL of the present invention is also shown: i.e., lens 426, plate-like haptic 427, and two capsular stabilizers 425a-b holding the peripheral circumferential edges of the capsule 424. It is noted that FIGS. 42a-schematically illustrate a cross section of the human eye with an IOL in a typical orientation with the posterior capsule intact, FIGS. 42b-d schematically illustrate a cross section of the human eye with an IOL in a typical orientation in the case of a missing posterior capsule 430. FIGS. 43a-c schematically illustrate a cross section of the human eye with an IOL in a typical orientation with the posterior capsule intact 431.

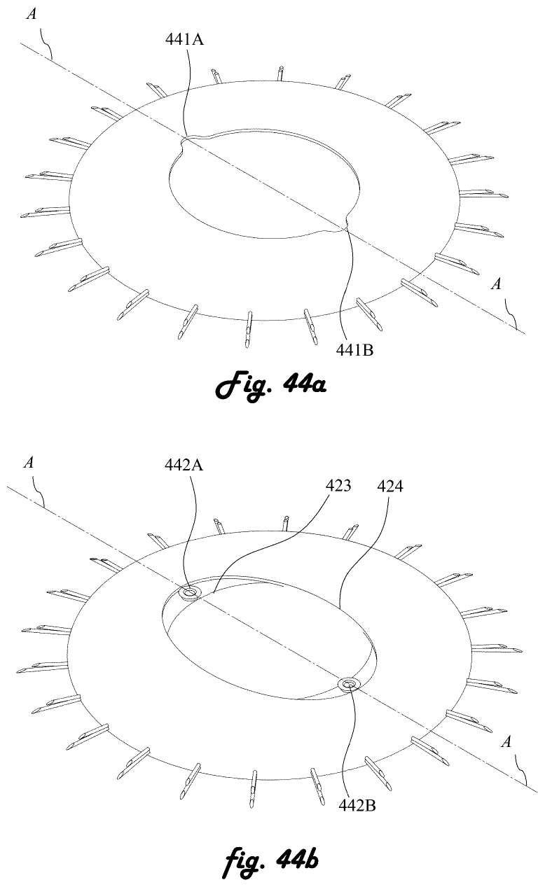

Reference is now made to FIGS. 44a-b schematically illustrate two approaches of marking the required positioning angle of a toric IOL by means of creating an opening in the anterior capsule preferably with the laser technology, according to a few embodiments of the invention: FIG. 44a depicts a technique of laser Capsulorhexis with notches positioned according to the required positioning angle of the toric lens and FIG. 44b depicts a technique of ellipse laser Capsulorhexis, with the long axis of the ellipse positioned according to the required positioning angle of the toric lens., e.g., indentations 441A-B. Capsulorhexis is a technique which has proved useful in removing the capsule of the lens from the eye during cataract surgery by shear and stretch forces, by a removal of a part of the anterior lens capsule, and, in situations like a developmental cataract, a part of the posterior capsule may also be removed by a similar technique. If a patient requires astigmatism correction along radian A:A, the surgeon marks in situ a premeasured astigmatism correction radian A:A by forming two notches. The surgeon further affixes ex situ a plurality of capsule stabilizing fasteners on the anterior surface along the axis B:B; so that capsule stabilizing heads protrude from the anterior surface X:Y so that a gap of a predefined size and shape is provided, facing the opposite side of the center of the lens. Then, the surgeon implants an IOL according to any embodiment of the present invention, namely an IOL comprising attached capsule stabilizers, with main longitudinal axis B:B (not shown here, see FIG. 7) positioned on an arbitrary radian. Subsequently, the surgeon in situ rotates the IOL comprising capsular stabilizers so that main longitudinal axis B:B is superimposed on the astigmatism radian A:A previously marked by notches 441A-B. Then, still in situ, the surgeon positions the capsule peripheral circumferential edge over the capsular stabilizers' gap to fixate the lens at the required angle and prevent its rotation and enable fixation to the anterior capsule.

In addition to toric IOL implantation, astigmatism can be corrected during and after cataract surgery, by either manual or femtosecond laser-assisted astigmatic keratotomy (FSAK), which is usually recommended for low-to-moderate astigmatism. FSAK uses a femtosecond laser to make arcuate, paired or unpaired partial-thickness incisions on a steep corneal meridian. FSAK can be used to create incisions with accurate angle, depth and location, which greatly improves the predictability and accuracy of corneal astigmatism correction compared to conventional manual astigmatic keratotomy, see e.g., Noh, Hoon, et al. “Comparison of penetrating femtosecond laser-assisted astigmatic keratotomy and toric intraocular lens implantation for correction of astigmatism in cataract surgery.” Scientific Reports 11.1 (2021): 1-10. It is hence in the scope of the invention wherein FSAK is used to perform a rhexis along a round, elliptical, or otherwise any predefined pattern, line, angle and shape so that capsular stabilizers according to any of the embodiments of the invention are positioned and effectively hold the IOL as required. FIG. 44b schematically illustrates two capsular stabilizers with anteriorly protruding heads 442A-B located along astigmatism radian A:A. An FSAK system is programmed to perform an ellipse Capsulorhexis 424. A rounded line 423 is shown for proportion.

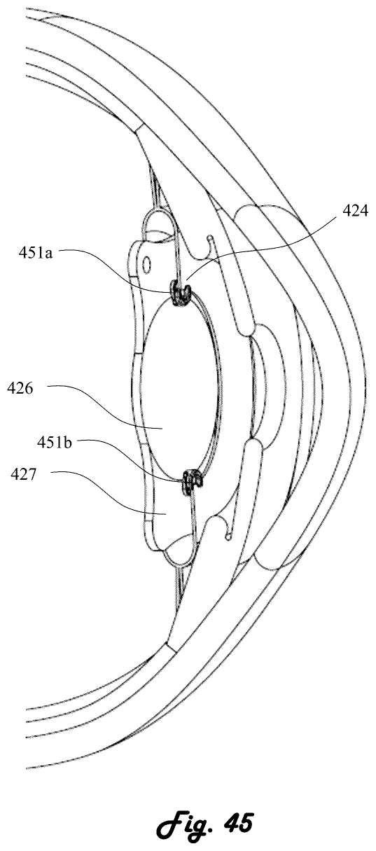

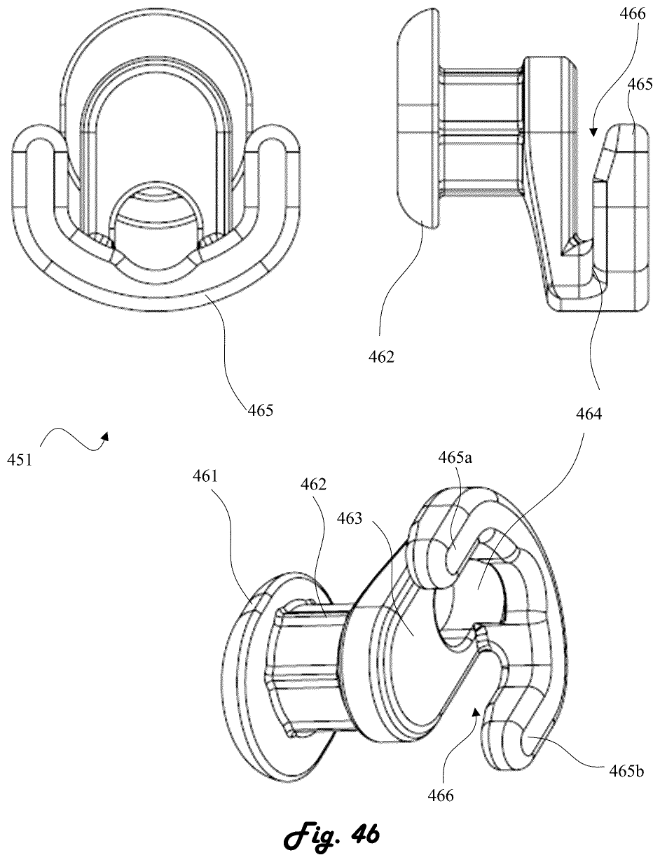

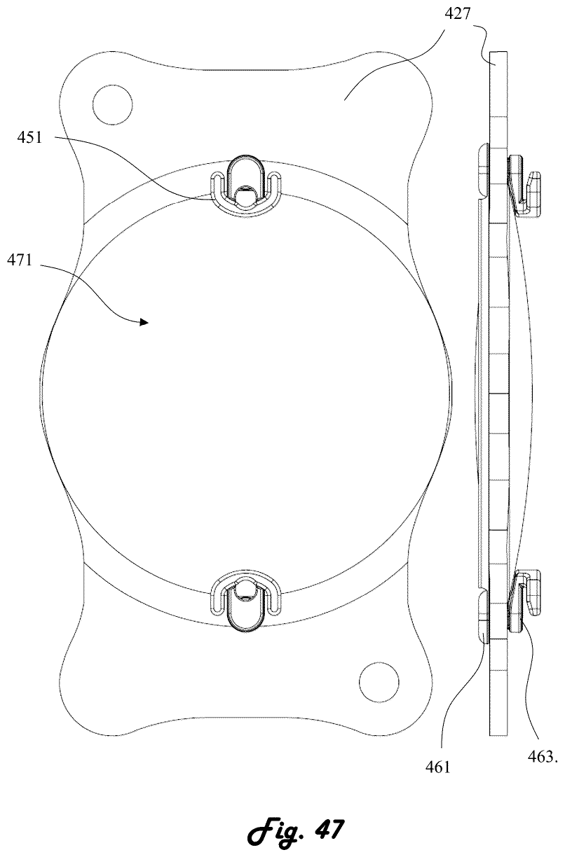

Reference is now made to FIG. 45-52. FIG. 45 schematically illustrates a cross section of an IOL implanted in a human eye. The IOL is having a lens (426), plate-type haptic 427, and two capsular stabilizers 451a-b holding the peripheral circumferential edges of the capsule 424. It is in the scope of the invention wherein capsular stabilizers 451a-b comprises a base portion 462 at haptic's posterior surface, interconnected with an anteriorly-protruding hook-like member 465, a two-fingers-like member (465a-b) via a stem member 462, see FIG. 46. The stem is incorporated within an aperture (e.g., hole 12A) across the width of the haptic. At the anterior surface of the haptic, a gap 466 is formed between plate 463 and the hook-like member 465, namely at neck 464. Each capsular stabilizer 451 is anchored to haptic 427, adjacent lens 471, see FIG. 47, so that base member 461 is located at the posterior surface of the haptic and the plate is located at the anterior surface of the haptic.

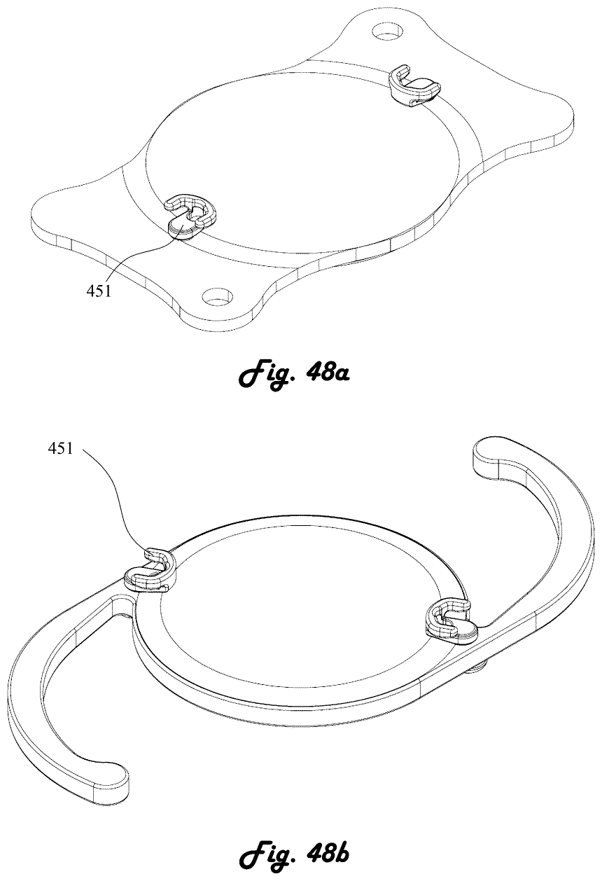

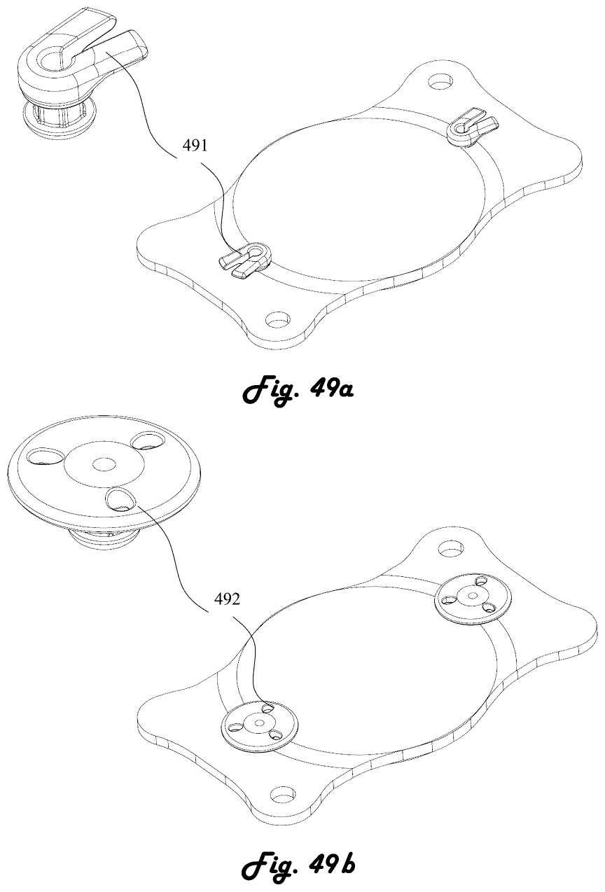

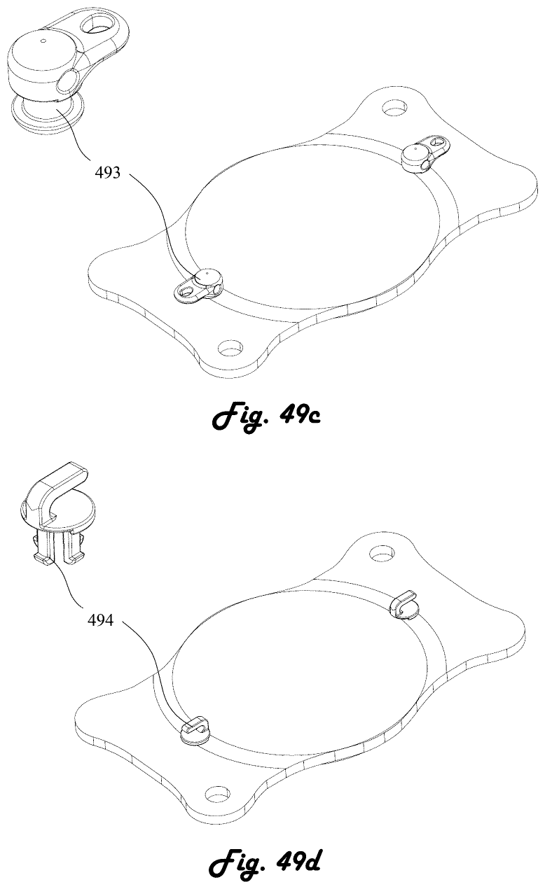

Reference is now made to FIGS. 48a and 48b, each of which is schematically illustrating a set of capsular stabilizers 451 incorporated in a plate-type haptic and a loop-type haptic, accordingly. FIGS. 49a-d schematically depict four different capsular stabilizers 491-494 according to a few embodiments of the invention: a V-shape capsule stabilizer, a button-like capsule stabilizer, a wide single-hook capsule stabilizer with an aperture, and a thinner single-hook capsule stabilizer, accordingly.

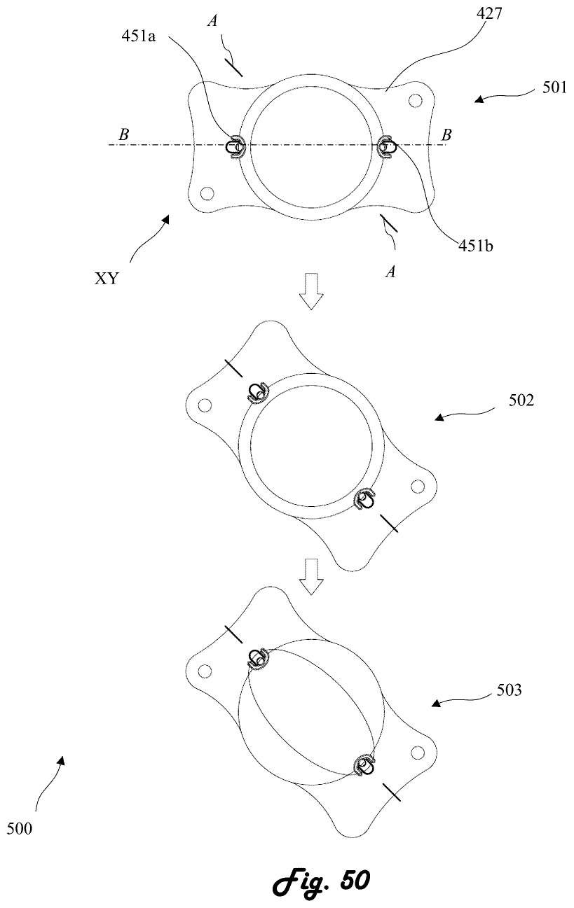

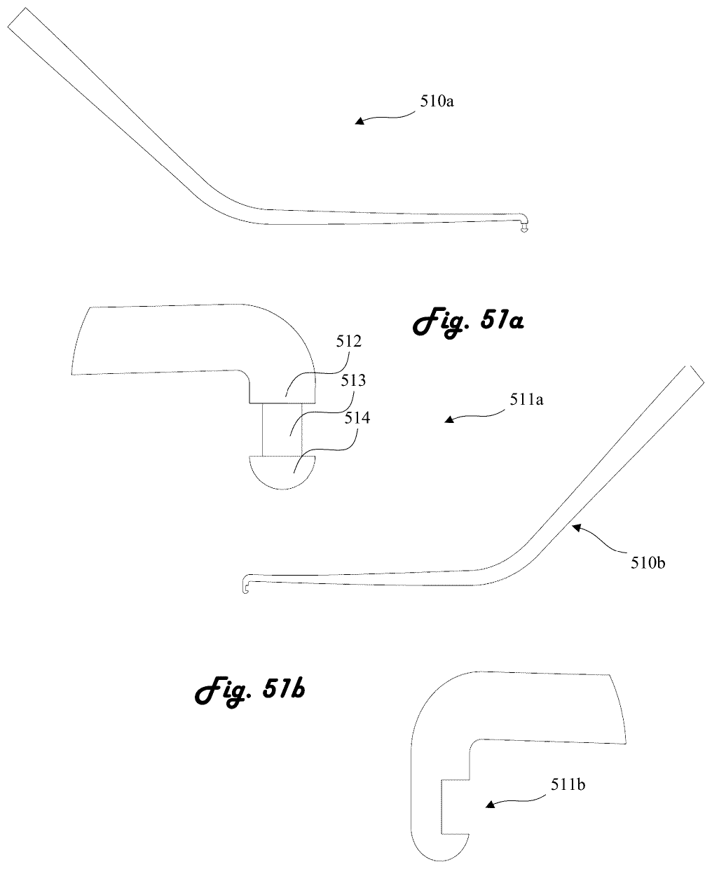

Reference is now made to FIG. 50, schematically showing three steps in a method (500) of implanting capsular stabilizers-containing haptic. In the example disclosed in FIG. 50, astigmatism correction is required along radian A:A. In one step (501), two capsular stabilizers (451a-b) are ex situ affixed, along a main longitudinal axis B:B of the haptic; so that the anteriorly protruding head portions of stabilizers are protruding the anterior surface X:Y and a gap of a predefined size and shape is provided, facing an opposite side of a center of said lens. In one or several steps (502), IOL is implanted and main longitudinal axis B:B is positioned on an arbitrary radian; the IOL is rotated in situ so that main longitudinal axis B:B is superimposed on said astigmatism radian A:A. In another step (503) a capsule peripheral circumferential edge is in situ positioned over the gap to fixate the lens at a required angle and to prevent its rotation and enable fixation to the capsule.

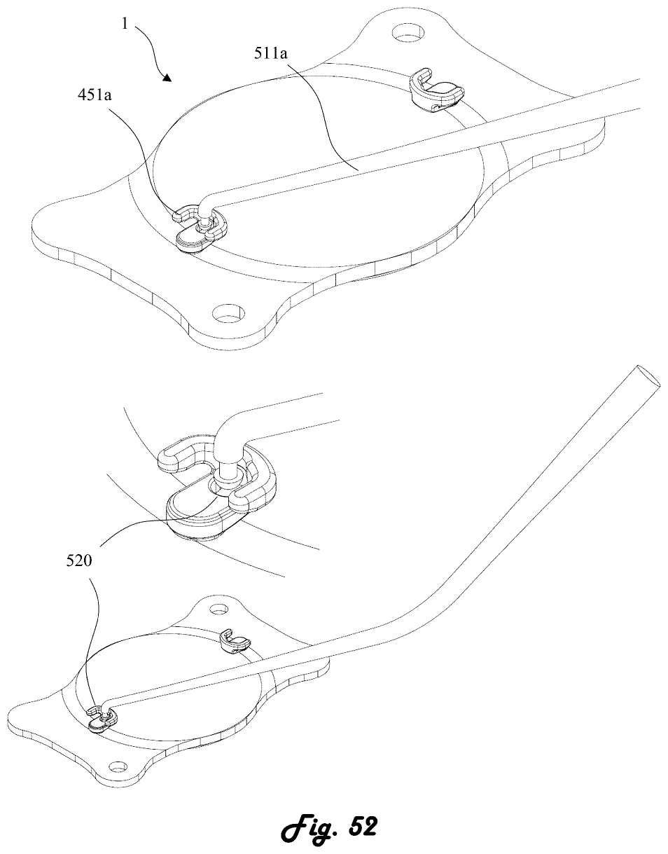

Reference is now made to FIGS. 51a-b and 52, schematically disclosing tools, modules, a medical device, a kit and methods for implanting capsular-stabilizers containing IOL 1 according to a few embodiments of the invention. Two tools (510a-b) for incorporation of capsular stabilizers within an IOL are shown. Both tools comprise a lever-like handle, interconnected with capsular stabilizers anchoring mechanism 511a-b; each of which comprises, inter alia, a distal base (514) to be located at the posterior portion of the haptic, a stem 513 and an anteriorly-located portion 512. The use of those tools, modules, medical devices, and kits is further illustrated in FIG. 52. Tool (511a) is having a stud-like connecting portion (520) in its distal-most end. The stud is inserted within an aperture at capsular stabilizer 451a, thereby it is ex situ affixing the capsular stabilizer to the haptic in a predefined location and orientation.

All publications mentioned herein are incorporated herein by reference. Nothing herein is to be construed as an admission that the invention is not entitled to antedate such disclosure by virtue of prior invention. Although the invention has been described and pictured in an exemplary form with a certain degree of particularity, it should be understood that the present disclosure of the exemplary form has been made by way of example, and that numerous changes in the details of construction and combination and arrangement of parts and steps may be made without departing from the pith and merrow and scope of the invention as set forth in the claims hereinafter.

1.-45. (canceled)

46. A vertical capsular stabilizer (VCS) useful for at least one of increasing postoperative rotation stability of an IOL; and enabling stabilization of an IOL within an anterior capsule for any indication, including in cases at which the posterior capsule is absent or ruptured, having a main longitudinal axis B:B, an anterior surface of a main plane X:Y and a posterior surface, comprising a lens interconnected to at least one haptic; said VCS is affixable perpendicular to said anterior surface along said main longitudinal axis B:B; when affixed, a head of said VCS protrudes from said anterior surface X:Y so that a gap of a predefined size and shape is provided.

47. The vertical capsular stabilizer of claim 46, wherein said lens is either a toric lens or a non-toric lens.

48. The vertical capsular stabilizer of claim 46, wherein said at least one haptic is selected from a group consisting of a plate-type haptic, a loop-type haptic and any combination thereof.

49. The vertical capsular stabilizer of claim 46, wherein said plurality of fasteners comprises n fasteners, n being equal to or greater than 2.

50. The vertical capsular stabilizer of claim 46, wherein said IOL further comprises a member of a group consisting of (i) at least one vertical hole; (ii) at least one horizontal hole; (iii) at least one vertically protruding structure; (iv) at least one horizontally protruding structure, and any combination thereof.

51. The vertical capsular stabilizer of claim 46, wherein said at least one VCS is selected from a group consisting of a symmetric snap, an asymmetric snap, a jeans-button anchor, a hook-containing member a loop-containing member, a hot-melt snap, a toothed clasp, a spring fastener and any combination thereof.

52. An IOL, characterized by at least one of an increased postoperative rotation stability of an IOL; and an increased stabilization within an anterior capsule for any indication, including in cases at which the posterior capsule is absent or ruptured, having a main longitudinal axis B:B, an anterior surface of a main plane X:Y and a posterior surface, comprising a lens interconnected to at least one haptic, a vertical capsular stabilizer of claim 1, reversibly affixable perpendicular to said anterior surface along said main longitudinal axis B:B; when affixed, a fastener head of said VCS protrudes from said anterior surface X:Y so that a gap of a predefined size and shape is provided, facing an opposite side of a center of said lens.

53. The IOL of claim 52, wherein said lens is either a toric lens or a non-toric lens.

54. The IOL of claim 52, wherein said at least one haptic is selected from a group consisting of a plate-type haptic, a loop-type haptic and any combination thereof.

55. The IOL of claim 52, wherein said plurality of fasteners comprises n fasteners, n being equal to or greater than 2.

56. The IOL of claim 52, wherein said IOL further comprises a member of a group consisting of (i) at least one vertical hole; (ii) at least one horizontal hole; (iii) at least one vertically protruding structure; (iv) at least one horizontally protruding structure, and any combination thereof.

57. The IOL of claim 52, wherein said at least one VCS is selected from a group consisting of a symmetric snap, an asymmetric snap, a jeans-button anchor, a hook-containing member a loop-containing member, a hot-melt snap, a toothed clasp, a spring fastener and any combination thereof.

58. A kit, that adds Capsular Fasteners to an IOL that is then characterized by at least one of an increased postoperative rotation stability of an IOL; and an increased stabilization within an anterior capsule for any indication, including in cases at which the posterior capsule is absent or ruptured, said kit comprising
a. an IOL having a main longitudinal axis B:B, an anterior surface of a main plane X:Y and a posterior surface, comprising a lens interconnected to at least one haptic; and
b. at least one vertical capsular stabilizer (VCS) of claim 1, reversibly affixable perpendicular to said anterior surface along said main longitudinal axis B:B; when affixed, a fastener head of said at least one VCS protrudes from said anterior surface X:Y so that a gap of a predefined size and shape is provided, facing an opposite side of a center of said lens.

59. The kit of claim 58, wherein at least one of the following is held true:
a. said lens is either a toric lens or a non-toric lens;
b. said at least one haptic is selected from a group consisting of a plate-type haptic, a loop-type haptic and any combination thereof;
c. said plurality of fasteners comprises n fasteners, n being equal to or greater than 2;
d. said IOL further comprises a member of a group consisting of (i) at least one vertical hole; (ii) at least one horizontal hole; (iii) at least one vertically protruding structure; (iv) at least one horizontally protruding structure, and any combination thereof; and
e. said at least one VCS is selected from a group consisting of a symmetric snap, an asymmetric snap, a jeans-button anchor, a hook-containing member a loop-containing member, a hot-melt snap, a toothed clasp, a spring fastener and any combination thereof.

60. A method for fixating an IOL to an anterior capsule for any indication, including in cases at which the posterior capsule is absent or ruptured, comprising steps of:
a. providing an IOL having a main longitudinal axis B:B, an anterior surface of a main plane X:Y and a posterior surface, said IOL comprising a lens interconnected to at least one haptic;
b. affixing at least one vertical capsular stabilizer (VCS) perpendicular to said anterior surface along said main longitudinal axis B:B; when affixed, a head of said at least one VCS protrudes from said anterior surface X:Y so that a gap of a predefined size and shape is provided, facing an opposite side of a center of said lens;
c. if astigmatism correction is required along radian A:A, ex situ affixing a plurality of said at least one VCS on said anterior surface along said main longitudinal axis B:B; so that heads of said plurality of at least one VCS protrude from said anterior surface X:Y so that a gap of a predefined size and shape is provided, facing an opposite side of a center of said lens;
d. implanting said IOL, said main longitudinal axis B:B positioned on an arbitrary radian;
e. if required, rotating in situ said IOL comprising said plurality of said at least one VCS so that said main longitudinal axis B:B is superimposed on said astigmatism radian A:A; and
f. in situ positioning a capsule peripheral circumferential edge over the gap to fixate the lens at a required angle, prevent rotation of the lens and enable fixation to the anterior capsule.

61. The method of claim 60, wherein said lens is either a toric lens or a non-toric lens.

62. The method of claim 60, wherein said at least one haptic is selected from a group consisting of a plate-type haptic, a loop-type haptic and any combination thereof.

63. The method of claim 60, wherein said plurality of fasteners comprises n fasteners, n being equal to or greater than 2.

64. The method of claim 60, wherein said IOL further comprises a member of a group consisting of (i) at least one vertical hole; (ii) at least one horizontal hole; (iii) at least one vertically protruding structure; (iv) at least one horizontally protruding structure, and any combination thereof.

65. The method of claim 60, wherein said at least one VCS is selected from a group consisting of a symmetric snap, an asymmetric snap, a jeans-button anchor, a hook-containing member a loop-containing member, a hot-melt snap, a toothed clasp, a spring fastener and any combination thereof.
